(19) United States
(12) Patent Application Publication
(10) Pub. No.: US 2011/0301239 A1
(43) Pub. Date: Dec. 8, 2011

(54) USE OF NOVEL LIPID MEDIATORS TO INHIBIT ANGIOGENESIS

(75) Inventors: Charles N. Serhan, Needham, MA (US); Reza Dana, Newton, MA (US); Yiping Jin, Winchester, MA (US)

(73) Assignee: The Brigham and Women's Hospital, Inc., Boston, MA (US)

(21) Appl. No.: 12/989,626

(22) PCT Filed: Apr. 27, 2009
(86) PCT No.: PCT/US09/41759

§ 371 Date: Apr. 29, 2011

Related U.S. Application Data
(63) Continuation of application No. 61/047,881, filed on Apr. 25, 2008.

(51) Int. Cl. A61K 31/201; A61P 27/02; A61P 9/00

(52) U.S. Cl. 514/560

(57) ABSTRACT

The present invention is generally drawn to novel isolated therapeutic agents, termed resolvins, generated from the interaction between a dietary omega-3 polyunsaturated fatty acid (PUFA) such as eicosapentaenoic acid (EPA) or docosahexaenoic acid (DHA) oxygenases and the analgesic aspirin (ASA). Surprisingly, careful isolation of compounds generated from the combination of components in an appropriate environment provide di- and tri-hydroxy containing derivatives of EPA or DHA containing compounds having unique structural and physiological properties. The present invention therefore provides for many new useful therapeutic di- and tri-hydroxy derivatives of EPA or DHA (resolvins of the E series and D series) that diminish, prevent, or eliminate NV, hemangiogenesis and/or angiogenic condition(s) of corneal tissue. The present invention also provides methods of use, methods of preparation, and packaged pharmaceuticals for use as medicaments for the compounds disclosed throughout the specification.

### CROSS REFERENCE TO RELATED APPLICATIONS

This application claims the priority date of U.S. Provisional Patent Application No. 61/047,881, filed Apr. 25, 2008. The disclosure of which is incorporated by reference herein.

### STATEMENT REGARDING FEDERALLY SPONSORED RESEARCH

The work leading to this invention was supported in part by Department of Defense Grant W81XWH-07-2-0038, NIH R01-EY 12963, NIH/NCRR P20 RR20753 Planning Grant For Research on Blinding Eye Diseases, NIH GM38675 and P50 DE0169191. The U.S. Government therefore may have certain rights in the invention.

### FIELD OF THE INVENTION

The present invention relates to previously unknown therapeutic agents derived from novel signaling and biochemical pathways that use eicosapentaenoic acid (EPA) and/or docosahexaenoic acid (DHA), which are polyunsaturated fatty acids (PUFAs, omega-3) as precursors to the production of bioactive novel endogenous products that control physiologic events in inflammation and resolution in vascular endothelial reactions and neural systems (brain). More specifically, the present invention relates to di- and trihydroxy potent bioactive products termed “Resolvins” and “Protectins” which are derived from polyunsaturated fatty acids. In addition, therapeutic stable analogs of resolvins of the E and D series and protectins that could enhance their biologic properties are described that can be used to expedite resolution by inhibiting the pro-inflammatory amplification of leukocyte entry.

### BACKGROUND OF THE INVENTION

The normal cornea has no blood or lymphatic vessels. This feature is essential for corneal transparency and optimal visual performance, and contributes to the immunologic privilege of the cornea.

Neovascularization (NV) is a common complication secondary to various corneal diseases, including infection, degeneration, trauma and stem cell deficiency-induced insults. NV is also strongly associated with graft failure after corneal transplantation. Additionally, corneal NV as a result of viral or chlamydial (trachoma) infection is a leading cause of visual impairment worldwide.

Corneal NV is a complex response to a number of stimuli, and involves a sequence of coordinated cellular and molecular mechanisms. Dilation of the existing limbal vessels followed by adhesion and diapedesis of leukocytes, such as neutrophils and macrophages, and migration and proliferation of vascular endothelial cells (EC), in large part mediated by VEGF, are all important factors in NV pathogenesis (1, 2, 3).

Limited therapeutics are available to topically treat inflammation in the cornea that are also able to regulate unwanted neovascularization of the corneal tissue. Current anti-inflammatories for topical treatments in the eye, i.e., applied directly to the cornea, include steroids, which are well appreciated by the clinical community to have long-term deleterious side effects. Such side effects include well-known complications such as cataracts, infection and glaucoma.

A need therefore exists for an improved understanding of neovascularization as well as the isolation and preparation of bioactive agents that can serve to eliminate or diminish NV pathogenesis, especially associated with the cornea.

### BRIEF SUMMARY OF THE INVENTION

The present invention, in one embodiment, is drawn to isolated therapeutic agents generated from the interaction between a dietary omega-3 polyunsaturated fatty acid (PUFA) such as eicosapentaenoic acid (EPA) or docosahexaenoic acid (DHA, an oxygenase, such as cyclooxygenase-II (COX-2), and an analgesic, such as aspirin (ASA). Surprisingly, careful and challenging isolation of previously unknown and unappreciated compounds are generated from exudates by the combination of components in an appropriate environment to provide di- and tri-hydroxy EPA and DHA derivatives having unique structural and physiological properties. The present invention therefore provides for many new useful therapeutic di- and tri-hydroxy derivatives of EPA or DHA that treat, prevent, or reduce NV, hemangiogenesis and/or angiogenesis.

Resolvins, such as resolvin E1 (RvE1; 5S,12R,18R-trihydroxyeicosapentaenoic acid) are novel anti-inflammatory lipid mediators derived from omega-3 fatty acid eicosapentaenoic acid (EPA). At the local site of inflammation, aspirin treatment enhances EPA conversion to 18R-oxygenated products including RvE1 that carry potent anti-inflammatory signals. Surprisingly, resolvins (the compounds identified throughout the specification) such as RvE1 protected against, reduced or inhibited the development of NV, hemangiogenesis and/or angiogenesis, in a well appreciated experimental mouse model.

The beneficial effect was reflected by decreased generation or elimination of neovascularization. Thus, the novel endogenous lipid mediators termed “resolvins”, such as RvE1 and NPD1 counterregulate leukocyte-mediated tissue injury and pro-inflammatory gene expression. These findings show a novel endogenous mechanism that may underlie the beneficial actions of omega-3 EPA and provides new approaches for the treatment of undesirable NV, hemangiogenesis or angiogenic conditions in the cornea.

The di- and tri-hydroxy EPA and DHA therapeutic agents of the invention useful to treat those indications noted throughout the specification, including those agents detailed throughout the specification numbered I through LXXX, for example:

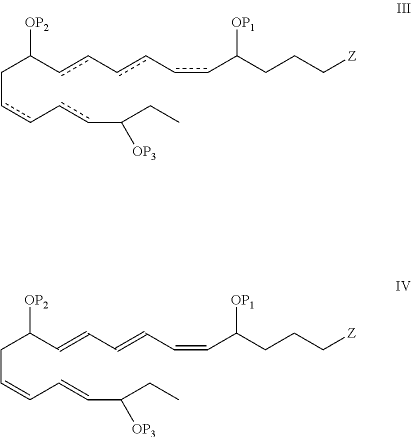

wherein a bond depicted as [symbol] represents either a cis or trans double bond;

wherein P₁, P₂ and P₃, if present, each individually are protecting groups, hydrogen atoms or combinations thereof;

wherein R₁, R₂ and R₃, if present, each individually are substituted or unsubstituted, branched or unbranched alkyl groups, substituted or unsubstituted aryl groups, substituted or unsubstituted, branched or unbranched alkylaryl groups, halogen atoms, hydrogen atoms or combinations thereof;

wherein Z is —C(O)OR^{d}, —C(O)NR^{c}R^{c}, —C(O)H, —C(NH)NR^{c}R^{c}, —C(S)H, —C(S)OR^{d}, —C(S)NR^{c}R^{c}, —CN;

each R^{a}, if present, is independently selected from the group consisting of hydrogen, (C1-C6) alkyl, (C3-C8) cycloalkyl, cyclohexyl, (C4-C11) cycloalkylalkyl, (C5-C10) aryl, phenyl, (C6-C16) arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;

each R^{b}, if present, is a suitable group independently selected from the group consisting of ═O, —OR^{d}, (C1-C3) haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙ—NR^{c}R^{c}, —[NHC(NH)]ₙNR^{c}R^{c} and —[NR^{a}C(NR^{a})]ₙNR^{c}R^{c};

each R^{c}, if present, is independently a protecting group or R^{a}, or, alternatively, each R^{c} is taken together with the nitrogen atom to which it is bonded to form a 5 to 8-membered cycloheteroalkyl or heteroaryl which may optionally include one or more of the same or different additional heteroatoms and which may optionally be substituted with one or more of the same or different R^{a} or suitable R^{b} groups;

each n, independently, if present, is an integer from 0 to 3;

each R^{d}, independently, if present, is a protecting group or R^{a};

in particular, Z is a carboxylic acid, ester, amide, thiocarbamate, carbamate, thioester, thiocarboxamide or a nitrile;

wherein X, if present, is a substituted or unsubstituted methylene, an oxygen atom, a substituted or unsubstituted nitrogen atom, or a sulfur atom;

wherein Q, if present, represents one or more substituents and each Q individually, if present, is a halogen atom or a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, amino, hydroxy, cyano, carboxyl, alkoxycarbonyloxy, aryloxycarbonyloxy or aminocarbonyl group;

wherein U, if present, is a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, alkoxycarbonyloxy, and aryloxycarbonyloxy group;

and pharmaceutically acceptable salts thereof.

In certain embodiments, Z is a pharmaceutically acceptable salt of a carboxylic acid, and in particular is an ammonium salt or forms a prodrug.

In certain embodiments, P₁, P₂, and P₃, if present, each individually are hydrogen atoms and Z is a carboxylic acid or ester. In other embodiments, X is an oxygen atom, one or more P's are hydrogen atoms, and Z is a carboxylic acid or ester. In still other embodiments, Q is one or more halogen atoms, one or more P's are hydrogen atoms, and Z is a carboxylic acid or ester.

In certain embodiments, R₁, R₂ and R₃, if present, are each individually lower alkyl groups, such as methyl, ethyl, and propyl and can be halogenated, such as trifluoromethyl. In one aspect, at least one of R₁, R₂ and R₃, if present, is not a hydrogen atom. Generally, Z is a carboxylic acid and one or more P's are hydrogen atoms.

In certain embodiments, when OP₃ is disposed terminally within the resolvin analog, the protecting group can be removed to afford a hydroxyl. Alternatively, in certain embodiments, the designation of OP₃ serves to denote that the terminal carbon is substituted with one or more halogens, i.e., the terminal C-18, C-20, or C-22 carbon, is a trifluoromethyl group, or arylated with an aryl group that can be substituted or unsubstituted as described herein. Such manipulation at the terminal carbon serves to protect the resolvin analog from omega P₄₅₀ metabolism that can lead to biochemical inactivation.

In certain embodiments, P₁, P₂, and P₃, if present, each individually are hydrogen atoms and Z is a carboxylic ester. In other embodiments, P₁, P₂, and P₃, if present, each individually are hydrogen atoms and Z is not carboxylic acid.

In one aspect, the compounds described herein are isolated and/or purified, in particular, compounds in which P₁, P₂, and P₃, if present, each individually are hydrogen atoms and Z is a carboxylic acid, are isolated and or purified.

In certain aspects of the invention, particular compounds are not included; these include the even numbered compounds identified above by Roman numbers, i.e., II, IV, VI, VIII, X, etc. through LXXX.

In one aspect, the resolvins described herein that contain epoxide, cyclopropane, azine, or thioazine rings within the structure also serve as enzyme inhibitors that increase endogenous resolvin levels in vivo and block “pro” inflammatory substances, their formation and action in vivo, such as leukotrienes and/or LTB₄.

Another embodiment of the present invention is directed to pharmaceutical compositions of the novel compounds described throughout the specification useful to treat or prevent NV, hemagenesis and/or angiogenesis of the cornea.

The present invention also provides methods to treat or prevent various disease states and conditions described throughout the specification, including for example, NV, hemagenesis and/or angiogenesis of the cornea.

The present invention further provides various methods to prepare the novel compounds described throughout the specification.

The present invention also provides packaged pharmaceuticals that contain the novel di- and tri-hydroxy EPA and DHA derivatives described throughout the specification for use in treatment with various NV, hemagenesis and/or angiogenesis of the corneal tissue.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1. Expression of receptors: ChemR23 in Inflamed Corneas. A. RT-PCR was used to analyze expression of ChemR23 (RvE1 receptor). Corneas of normal or inflamed eyes (10 corneas pooled per group) were collected and the epithelium was subsequently separated from the subjacent stroma-endothelium in the respective groups. RNA was isolated from these tissues, as well as from MK/T-1 cells (corneal keratocyte cell line) with or without TNF-α and IL-1β stimulation, or FACS-sorted CD11b⁺ cells from inflamed corneas. In addition, RNA was isolated from lymph nodes as a positive control. This experiment was repeated 3 times. B. The mean density of each band was measured by using NIH image J software. The density of the ChemR23 band were normalized with the density of the corresponding GAPDH band. Data is shown as a mean of 3 experiments, and error bars represent SEM.

FIG. 2. Resolvins Reduce Neutrophil and Macrophage Infiltration in Inflamed Corneas. RvD1, RvE1, or vehicle was subconjunctivally injected at 0 h and 48 h after suture placement. For each compound treatment, eyes were enucleated from a group of mice at 24 h, and another group at 72 h after suture placement (3 eyes per group). Cross-sections were stained with anti-neutrophil (NIMP-R14) or anti-macrophage (F4/80) Ab, and 12 sections were used to enumerate the respective leukocyte populations. Results represent the mean (±SEM) of 3 eyes per group (*P<0.05, **P<0.001 vs vehicle-treated group, t-test), and data are representative of two independent experiments.

FIG. 3. Resolvins Reduce Cytokine mRNA Expressions in Inflamed Corneas. RvD1, RvE1, or vehicle was subconjunctivally injected at 0 h and 48 h after suture placement. For each compound treatment, corneas were harvested from a group of mice at 24 h, and another group at 72 h after suture placement, as well as from normal untreated control corneas (6 corneas per group). mRNA levels of inflammatory cytokines (including IL-1α, IL-1β, and TNF-α) were determined by real-time PCR. Data were normalized to GAPDH mRNA and values were expressed as the fold change over normal control corneas. Results represent the mean (±SEM) of three samples per group (each sample consisted of 2 pooled corneas), and data are representative of two independent experiments (*P<0.05, **P<0.001 vs vehicle-treated group, t-test).

FIG. 4. The Impact of Resolvins on the mRNA Expression of VEGFs and VEGFRs in Inflamed Corneas. RvD1, RvE1, or vehicle was subconjunctivally injected at 0 h and 48 h after suture placement. For each compound treatment, corneas were harvested from a group of mice at 24 h, and another group at 72 h after suture placement, as well as from normal untreated control corneas (6 corneas per group). (A) VEGF ligand species (VEGF-A, VEGF-C, VEGF-D) and (B) VEGFRs (VEGFR2 and VEGFR3) were tested by real-time PCR and normalized to GAPDH mRNA. Values are expressed as fold change over the normal control cornea. Results represent the mean (±SEM) of three samples per group (each sample consisted of 2 pooled corneas), and the data are representative of two independent experiments (*P<0.05, **P<0.001, ***P<0.0005, vs vehicle-treated group, t-test).

FIG. 5. Suture-induced Corneal HA is Reduced with Resolvins. RvD1, RvE1, or vehicle was subconjunctivally injected every 48 h from 0 to 14 days after suture placement. A. In a masked fashion, corneal NV was scored biomicroscopically with a slit-lamp using a grid system. Values are expressed as the mean (±SEM) of 6 corneas. B. Whole corneal tissues were harvested on day 14 and double-stained with anti-CD31 (green) and anti-LYVE-1 (red) for epifluorescence microscopy (20× magnification). C. The density of blood vessels (CD31^{high}/LYVE-1⁻) or lymphatic vessels (CD31^{low}LYVE-1^{high}) covering the cornea was analyzed. Values are expressed as the mean (±SEM) of 6 corneas of per treatment group (**P<0.001 vs vehicle-treated group, t-test), and the data are representative of two independent experiments.

FIG. 6. RvD1 and RvE1 Regulate IL-1β- and VEGF-A-induced Corneal HA and LA. Hydron pellets containing IL-1β or VEGF-A were implanted into the corneas on day 0. RvD1, RvE1, or vehicle was subconjunctivally injected every 48 h from 0 to 7 days after pellet implantation (4 corneas per treatment, for each pellet-type). A. Slit-lamp examination was performed on day 7 and representative images are shown. B. The density of blood vessels (CD31^{high}/LYVE-1⁻) or lymphatic vessels (CD31^{low}LYVE-1^{high}) covering each cornea was analyzed. Values are expressed as the mean (±SEM) of each treatment group (4 corneas per group) and the data are representative of two independent experiments (*P<0.05 vs vehicle-treated group, t-test).

### DETAILED DESCRIPTION

The features and other details of the invention will now be more particularly described and pointed out in the claims. It will be understood that the particular embodiments of the invention are shown by way of illustration and not as limitations of the invention. The principle features of this invention can be employed in various embodiments without departing from the scope of the invention.

In the specification and in the claims, the terms “including” and “comprising” are open-ended terms and should be interpreted to mean “including, but not limited to . . . .” These terms encompass the more restrictive terms “consisting essentially of” and “consisting of:

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

Terms and abbreviations used throughout the specification include:

ASA, aspirin

BV, blood vessels

COX, cyclooxygenase

EC, endothelial cells

EPA, eicosapentaenoic acid

DHA, docosahexaenoic acid

DSS, dextran sodium sulfate

GC-MS, gas chromatography-mass spectrometry

HA, hemoangiogenesis or hemangiogenesis

7S,17R-dihydroxy-DHA, 7S,17R-dihydroxy-docosa-4Z,8E,10Z,13Z,15E,19Z-hexaenoic acid

4S,17R-dihydroxy-DHA, 4S-17R-dihydroxy-docosa-5E,7Z,10Z,13Z,15E,19Z-hexaenoic acid

7S,17R,22-trihydroxy-DHA, 7S,17R,22-trihydroxy-docosa-4Z,8Z,10Z,13Z,15E,19Z-hexaenoic acid

4S,11,17R-trihydroxy-DHA, 4S,11,17S,-trihydroxy-docosa-5E,7E,9Z,13Z,15E,19Z-hexaenoic acid

LA, lymphangiogenesis

LC-UV-MS-MS, liquid chromatography-UV diode array detector-tandem mass spectrometry

LO, lipoxygenase

LT, leukotriene

LV, lymphatic vessels

MPO, myeloperoxidase

NV, neovascularization

PDA, photodiode array detector

PUFA, polyunsaturated fatty acids

Rv, resolvin, resolution phase product

RvD 1, 7S,8R,17S-trihydroxy-4Z,9E,11E,13Z,15E,19Z-docosahexaenoic acid

RvE1, 5S,12R,18R-trihydroxy-6Z,8E,10E,14Z,16E-EPA

Lymphangiogenesis, an important initial step in tumor metastasis and transplant sensitization, is mediated by the action of VEGF-C and -D on VEGFR3. Lymphangiogenesis is the structural organization of the lymphatic vessels and their growth

Angiogenesis, the outgrowth of new from preexisting blood vessels, is an important pathogenic aspect of tumor growth, chronic inflammatory diseases, and most blinding ocular conditions. To clearly separate it from the process of lymphangiogenesis, it should be understood that blood vascular angiogenesis is referred to as hemangiogenesis (HA). In recent years, much has been learned about the stimulators and inhibitors of HA and lymphangiogenesis, and members of the VEGF family have emerged as prime mediators of both processes. The VEGF growth factor family consists of five members that bind to and activate three distinct receptors. VEGF-A binds to VEGFR1 and VEGFR2, and placental growth factor (PlGF) and VEGF-B bind only to VEGFR1. VEGF-C and VEGF-D bind to VEGFR2 and VEGFR3.

VEGF-A has emerged as the family member principally responsible for normal vasculogenesis and HA. The direct effects of VEGF-A on vascular endothelial cells are mediated principally via VEGFR2 ligation, while, until recently, VEGFR1 was thought to mediate mainly inhibitory or decoy functions. VEGF-A also plays a predominant role in diverse forms of pathological angiogenesis, including those requisite for the rapid growth of solid tumors. For this reason many antiangiogenic agents currently in development for the treatment of cancers have targeted VEGF-A or VEGFR2.

In contrast to HA, lymphangiogenesis is thought to be mediated mainly by the binding of VEGF-C and -D to their high-affinity receptor, VEGFR3. Like HA, lymphangiogenesis has gained much attention recently as an important initial step in tumor pathogenesis. It has been shown that intra- and/or peritumoral lymphangiogenesis increases the risk for metastasis both in animal models and in human tumors. The release of the lymphangiogenic growth factors VEGF-C and -D has been linked to a circulating subfraction of CD14+, VEGFR3-expressing monocytes that are recruited to and activated at the site of tumor growth.

Resolvins are lipid mediators generated from essential polyunsaturated fatty acids that are the first dual anti-inflammatory and pro-resolving signals identified in the resolution phase of inflammation. Here, the potential of resolvin D1 (RvD 1), and resolvin E1 (RvE1), in regulating angiogenesis in a corneal neovascularization (NV) murine system in vivo, in which sutures or micropellets containing IL-1β or VEGF-A were placed was investigated. First, it was determined that the receptor for RvE1, ChemR23, was expressed by the epithelium, stromal fibroblasts, and CD11b⁺ cells, in both normal and inflamed corneas. Mice were treated with either RvD1 or RvE1, subconjunctivally (100 ng/10 μl) at 48 h intervals. The control NV group was treated with vehicle only. RvD1- and RvE1-treated eyes had reduced numbers of infiltrating neutrophils and macrophages. The mRNA expression levels of TNF-α, IL-1α, IL-1β, VEGF, VEGF-C, and VEGFR2 were significantly reduced after treatment with these lipid mediators, compared to the control group. Animals treated with these mediators had significantly suppressed suture-induced or IL-1β-induced hemoangiogenesis (HA), but not lymphangiogenesis. These results indicate that RvD1 and RvE1, each reduce inflammatory corneal HA by early regulation of resolution mechanisms in innate immune responses.

The present invention provides that resolvins, such as D1 (RvD1) and resolvin E1 (RvE1 significantly reduce neutrophil and macrophage infiltration, accompanied by down-regulation in the expression of inflammatory cytokines and angiogenic growth factors and receptors. Moreover, these changes have a significantly greater effect in reducing hemoangiogenesis (HA) than lymphangiogenesis (LA).

### Results

### Resolvin E1 Receptor Expression in the Cornea

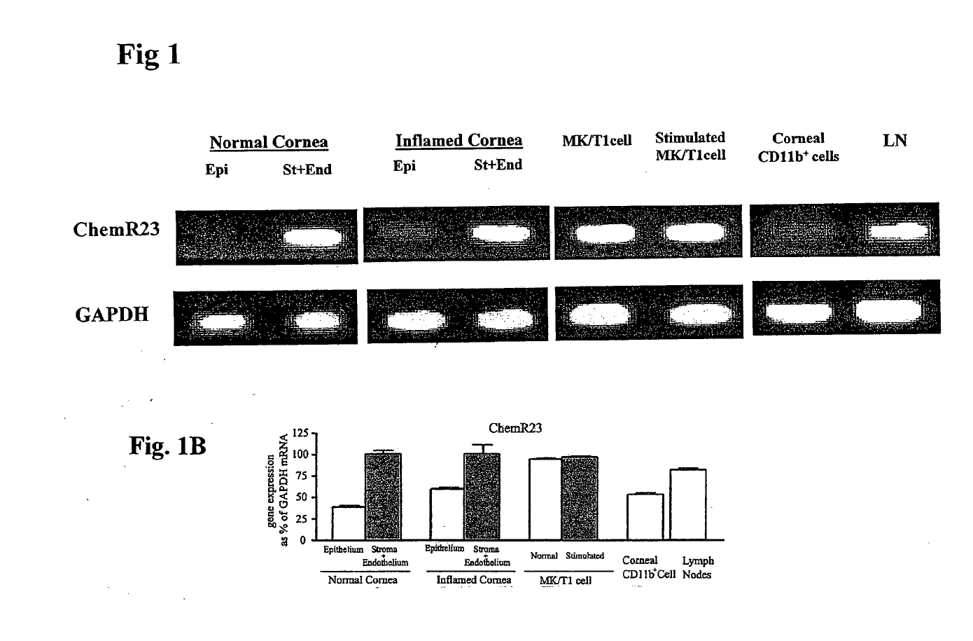

It was found that the expression of the receptor for RvE1 in corneal tissue by RT-PCR. In the normal corneas, ChemR23 (the RvE1 receptor), was present in both the epithelial and stromal-endothelial layers (FIG. 1). The receptor for RvD1 has not been identified to date.

To further delineate whether corneal keratocytes versus immunocytic CD11b⁺ cells (i.e., macrophages and dendritic cells) in the corneal stroma express these receptors, MK/T-1 cells (an immortalized corneal keratocyte cell line) stimulated with TNF-α and IL-1β were cultured to mimic in situ corneal inflammation. RT-RCR results showed ChemR23 was expressed by MK/T-1 cells irrespective of cytokine stimulation. In addition, the infiltrated CD11b⁺ cells, sorted from the inflamed corneas, also expressed high levels of ChemR23 (FIG. 1).

Application with RvD1 or RvE1 Reduces Neutrophil and Macrophage Infiltration

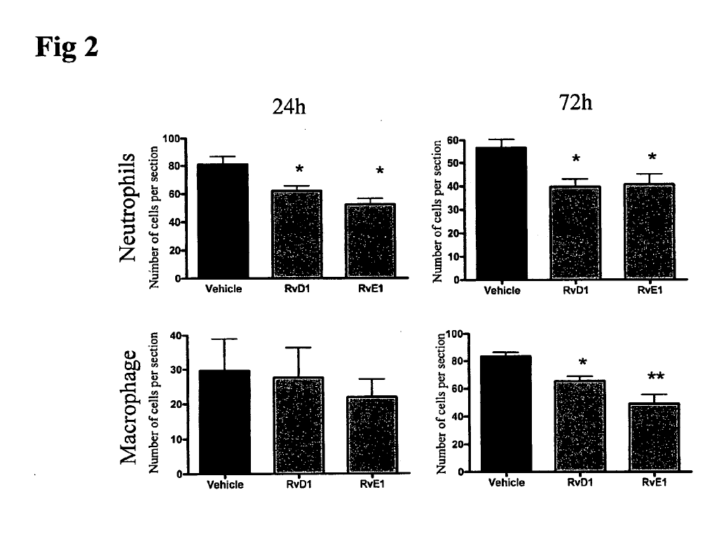

Next, inflamed corneas were treated with RvD1 or RvE1 to assess the action of these lipid mediators on the infiltration of neutrophils and macrophages (FIG. 2). An approximate 30% inhibition of neutrophils recruitment (p<0.05) into the inflamed corneas was observed at 24 and 72 h with the administration of RvD1 (neutrophils; 24 h: 62±4 cells/section, 72 h: 40±3 cells/section, n=3), or RvE1 (neutrophils; 24 h: 52±4 cells/section, 72 h: 41±4 cells/section, n=3), compared to the vehicle-treated group (neutrophils; 24 h: 81±6 cells/section, 72 h: 57±4 cells/section, n=3). Similarly, macrophage infiltration was also reduced, but this was only observed at 72 h after suture placement: RvD1 (macrophage; 57±4 cells/section, n=3), or RvE1 (macrophage; 49±7 cells/section, n=3) resulted in a 25-40% (p<0.05) reduction in macrophage infiltration compared to the vehicle control group (macrophage; 83±3 cells/section, n=3).

Application with RvD1 or RvE1 Reduces Inflammatory Cytokine Expression

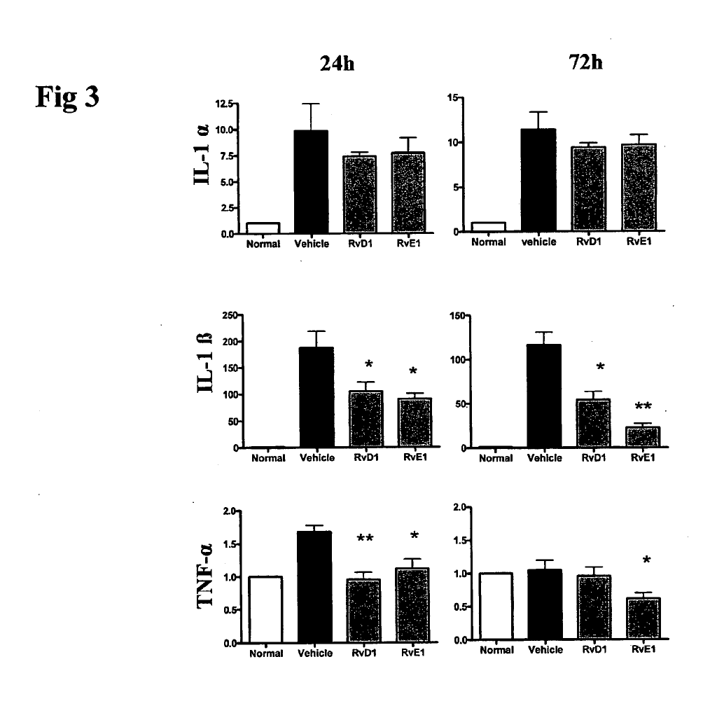

The mRNA levels of inflammatory cytokines, IL-1α, IL-1β, and TNF-α, were monitored by real-time PCR at 24 h and 72 h after suture placement (FIG. 3). Treatment with RvD1 or RvE1 led to a more than 50% reduction in the increase of IL-1β expression levels compared to the vehicle-treated controls (p<0.05) after induction of corneal inflammation. Increases in TNF-α expression were also significantly suppressed by these two mediators at 24 h, but not at 72 h, except for RvE1 application which led to a significant lowering of TNF-α expression at 72 h. The expression of IL-1α was not significantly altered by these lipid mediators (FIG. 3). The present invention also confirmed protein levels of inflammatory cytokine IL-1β using ELISA in the different treatment groups.

### RvD1 or RvE1 Impacts the Expression of VEGFs and VEGFRs

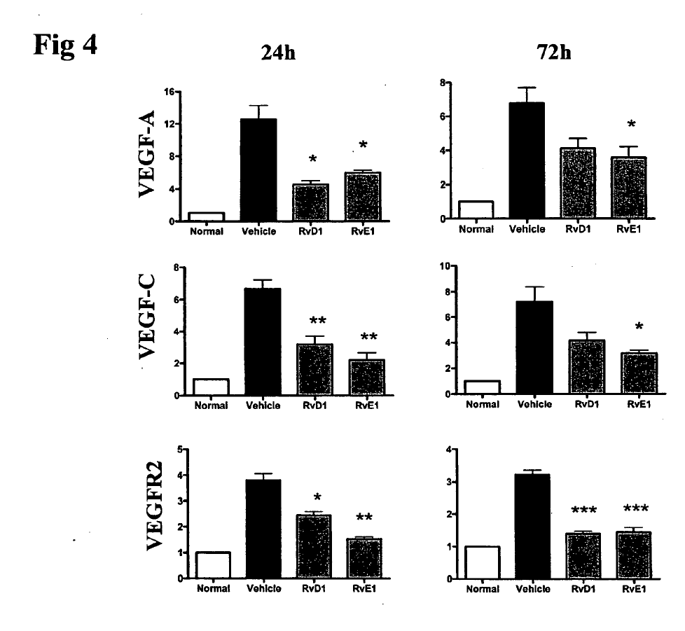

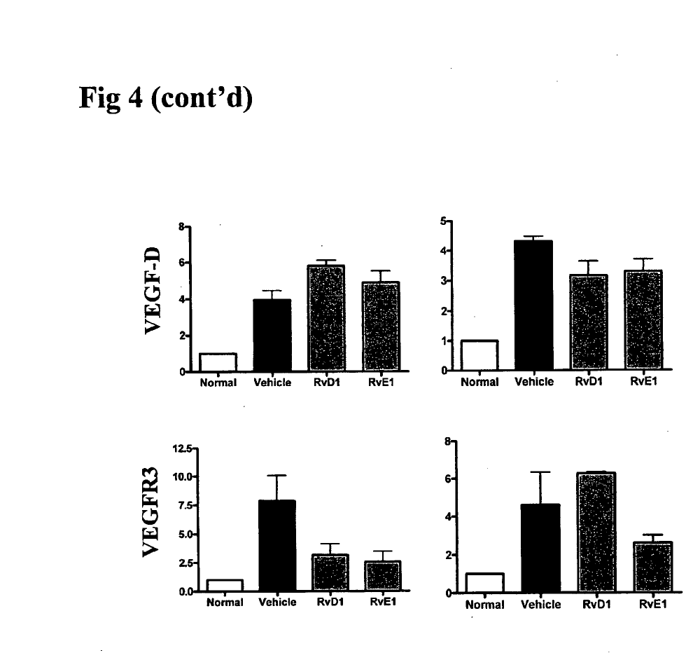

To determine the effect of RvD1 and RvE1 in modulation of angiogenesis, mRNA expression was measured for the critical ligands (VEGF-A, C, D) and receptors (VEGFR-2, 3) involved in angiogenesis (FIG. 4). In contrast to the vehicle-treated group, RvD1, and RvE1 application groups uniformly had lower mRNA expressions of the angiogenic growth factors, VEGF-A, C and their receptor, VEGFR-2, at 24 h and 72 h after suture placement. However, the mRNA expression for the lymphangiogeneic growth factor, VEGF-D and its receptor, VEGFR-3, were not significantly altered in the RvD 1 and RvE1 groups relative to the vehicle-treated controls.

### Evaluation of Clinical Corneal NV and Histological Assessment of HA and LA

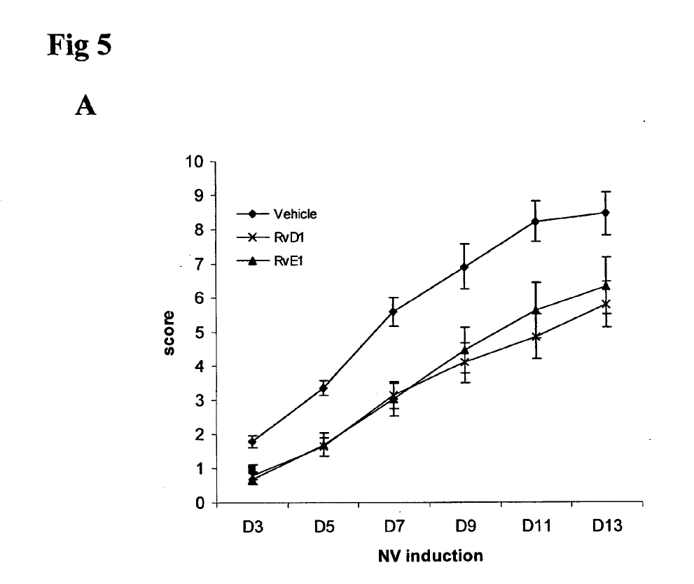

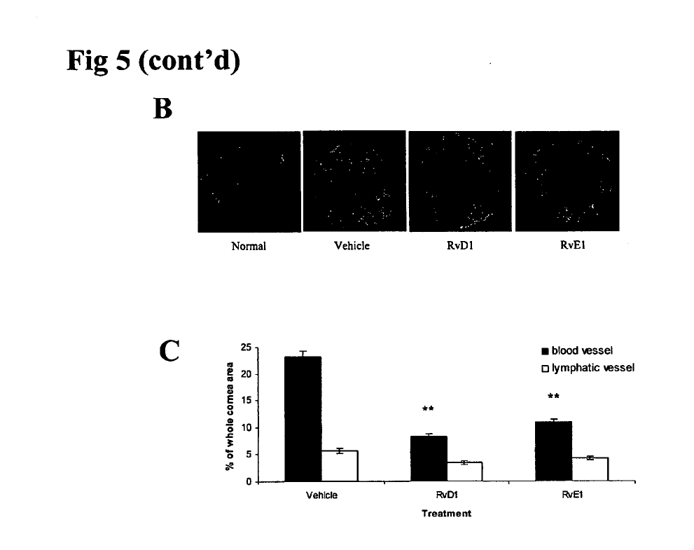

The growth of corneal neovessels was measured over a 2-week time course via slit lamp biomicroscopy. Use of corneal sutures in this model induces inflammatory NV within 2 days and peaks approximately 2 weeks post manipulation as described previously.¹⁴ It was observed that application with any of the two lipid mediators led to significant suppression of the angiogenic response, relative to the vehicle control (FIG. 5). It was further compared to the density of the blood vessels (BV) and lymphatic vessels (LV) using whole-mounted corneas harvested from the different groups, and co-stained these with anti-CD31 and anti-LYVE-1 (BV are CD31^{high}/LYVE-1⁻, while the LV are CD31^{low}LYVE-1^{high}). Consistent with slit-lamp observations, by day 14 after suture placement the BV density was significantly suppressed with RvD1 (8.4±0.39%, n=6) or RvE1 (10.92±0.53%, n=6) application, relative to vehicle application (23.18±1.12%, n=6). Interestingly, significant changes in the density of lymphatic vessels among the lipid mediator groups relative to the vehicle controls were not observed.

RvD1 or RvE1 Regulation of IL-β and VEGF-A induced-HA

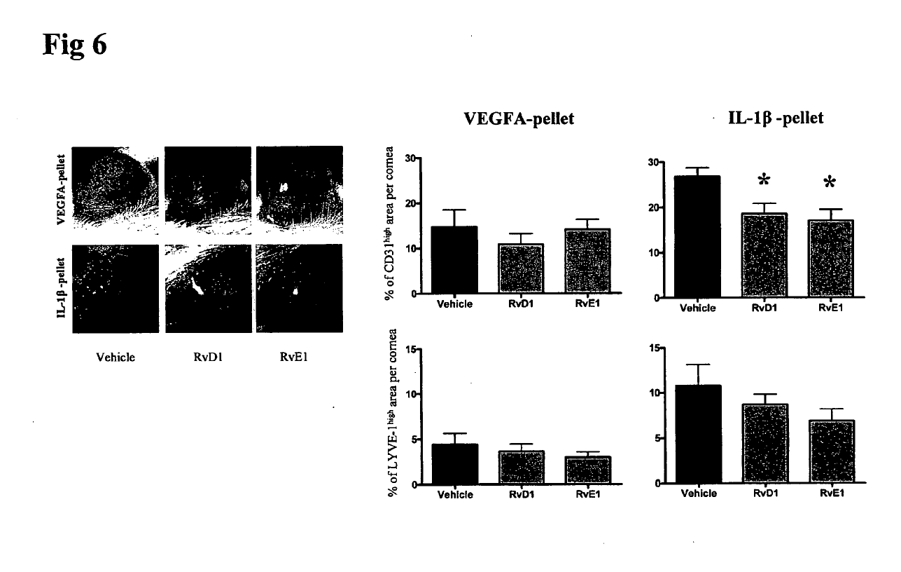

To further dissect the direct regulatory actions of these lipid mediators on VEGF-A-induced angiogenesis versus a more ‘indirect’ inhibitory effect on angiogenesis via suppression of innate immune responses, HA and LA were measured after intrastromal placement of micropellets loaded with IL-1β- or VEGF-A. Quantitative analysis of corneal flat-mounts harvested from VEGF-A micropellet stimulation showed that the BV density in the group treated with treated with RvD1 (10.9±2.3%, n=4) or RvE1 (14.1±2.2%, n=4), was not significantly lower relative to vehicle treatment (14.75±3.8%, n=4). Vessel growth stimulated by IL-1β-micropellets was more marked than that with VEGF-A stimulation. Nonetheless, treatment with either RvD1 (BV density; 17.1±2.4%, n=4) or RvE1 (BV density; 18.6±2.2%, n=4) significantly impaired IL-1β-induced BV growth, relative to vehicle treatment (BV density; 26.8±2.0%, n=4). Interestingly, and corroborating with previous observations in suture-induced corneal NV, no significant reduction of LA stimulated by either IL-1β- or VEGF-A was observed with any of these mediator treatments (FIG. 6).

### Discussion

The present invention provides that the lipid mediators, RvD1 and RvE1, regulate VEGF-A/-C and VEGFR2 and as a result, significantly reduce the development of NV in the inflamed cornea, in addition to their resolving effects on innate immunity. The present invention also provides that corneal tissues and infiltrating innate immune cells express ChemR23 (the RvE1 receptor); the ligation of which directly suppress angiogenesis. In the aggregate, these results in a model of surgically induced corneal inflammation and angiogenesis confirm the functions of RvD 1 and RvE1 as potent dual anti-inflammatory and pro-resolution molecules that can also effectively stop angiogenesis.

The neutrophil is the most prominent and earliest cell to migrate into the cornea in the early stages of inflammation, and anti-inflammatory lipid mediators can promote resolution by shortening the duration of neutrophil tissue infiltration.²⁵ In line with this current understanding, the present invention provides that the administration of RvD1 and RvE1, indeed blocked neutrophil infiltration of the cornea at 24 h and 72 h post insult—a time point which coincided with the down-regulation of proinflammatory cytokine (e.g., TNF-α, IL-1-α, and IL-1-β) expression known to be secreted by innate immunocytes, in particular neutrophils. Moreover, the data also show that macrophage infiltration is also reduced significantly after local administration of RvD1 and RvE1. These results highlight the importance of neutrophil infiltration in the local chemotaxis of subsequent immunocyte populations such as the macrophage. It has been shown that RvE1 can also increase macrophage phagocytosis (e.g. of apoptotic neutrophils) and this may also contribute to the resolution of inflammation following treatments.²⁶ Taken together, local administration of these lipid mediators to the cornea control local innate immune cell infiltration and enhance the resolution of inflammation.

While the healthy or normal cornea is avascular, local inflammation can stimulate the ingrowth of neovessels from the surrounding limbal and conjunctival areas through secretion of pro-angiogenesis factors by local vascular endothelial and inflammatory cells. Cytokines such as IL-1β, IL-1α, and TNF-α, are known to enhance the expression of angiogenic factors.²⁷⁻²⁹ Among all the angiogenic factors, the VEGF species play a pivotal role in vascular development. Ligation of VEGFR2 by VEGF-A is critical in vascular EC proliferation and differentiation in hemangiogenesis. On the other hand, binding of VEGF-C/-D to VEGFR3 stimulates the development of lymphatic vessels. In addition, VEGF-C can also bind and activate VEGFR2,^{30,31} and thereby contribute to HA, despite having a weaker binding affinity than VEGF-A.³² Interestingly, the present invention provides that treatments with RvD 1 or RvE1 significantly reduced the gene expression levels of VEGF-A, VEGF-C and their receptor VEGFR2, but not VEGF-D or VEGFR3. This indicates that such treatments selectively regulate HA, rather than LA, and this was further supported by immunohistochemistry.

While IL-1 secretion can stimulate VEGF expression and thereby promote angiogenesis, administration of exogenous VEGF-A can achieve a similar effect (possibly independent of other downstream effectors resulting from IL-1 signaling). It was therefore questioned whether the suppression of corneal NV by RvD1 and RvE1 is due to suppression of IL-1β and/or VEGF-A stimulation. Very recently, the contribution of RvD1 and RvE1 to the regulation of angiogenesis through the suppression of TNF-α expression has been reported based on a hypoxia-induced pathological retinal NV mouse model.³³ Here, the present invention provides that RvD 1 and RvE1 efficiently reduce IL-1β-induced, but not VEGF-A-induced, corneal angiogenesis. This suggests that the anti-angiogenesis function of RvD 1 and RvE1 largely depends on their anti-inflammatory/pro-resolution function, rather than direct regulation of VEGF-A function. This is further supported by a reduction of macrophage infiltration observed in IL-1β-induced corneal angiogenesis with RvE1 and RvD 1 treatment.³⁴

ChemR23, a receptor for RvE1 involved in attenuation of TNF-α activated NF-κB, is abundantly expressed in macrophages and dendritic cells, but less so in neutrophils.^{18,40} The present invention provides that ChemR23 is expressed by the infiltrating CD11b⁺ cells (which include macrophages, and monocytic dendritic cells, and a subset of neutrophils) in the cornea. These results suggest that RvE1 activation of ChemR23, on these CD11b⁺ immunocytes stops their local migration and cytokine production into cornea.

The present invention also provides for the distribution of ChemR23 in the cornea, which include epithelial cells and stromal keratocytes in both normal and inflammatory conditions. In conclusion, RvD1 and RvE1 effectively resolve corneal inflammation and angiogenesis by controlling innate inflammation via marked reduction of proinflammatory cytokine secretion and inhibiting VEGF/VEGFR expression. These novel lipid mediators offer a potentially new therapeutic strategy in controlling corneal angiogenesis, a leading cause of visual blindness worldwide.

### Methods

### Animals

Six to eight-week-old male BALB/c (Taconic Farms, Germantown, N.Y.) mice were used in all experiments. All experimental protocols were approved by the Schepens Eye Research Institute Animal Care and Use Committee, and all animals were treated according to the Association for Research in Vision and Ophthalmology Statement for the Use of Animals in Ophthalmic and Vision Research.

### Suture-induced Inflammatory Corneal Angiogenesis

The standard model for induction of inflammatory corneal NV is associated with development of intrastromal vessels in close association with a mixed-cell (primarily neutrophilic) infiltrate.^{14,15} Three interrupted sutures (11-0 nylon, Sharpoint; Vanguard, Houston, Tex.) were placed intrastromally with two stromal incursions extending over 120° of the corneal circumference each to induce inflammatory corneal NV, which is also associated with significant LA, as described previously.¹⁴ The corneas were followed by slit-lamp biomicroscopy for corneal NV development. NV was graded between 0 and 3, with increments of 0.5, using a grid system per each corneal quadrant based on the centripetal extent of the neovascular branches from the limbus. Scores for each quadrant then summed to derive the NV index (range 0 to 12) for each eye, as previously described.¹⁴

### Corneal Micropocket Assay

The corneal micropocket assay in mice and quantification of the resultant NV has been described previously.^{16,17} In brief, 0.3 μl of hydron pellets (IFN Sciences, New Brunswick, N.J.) containing 30 ng of murine IL-1β (R&D Systems, Minneapolis, Minn.) or 200 ng of VEGF-A (gift from BRB Preclinical Repository, National Cancer Institute) were prepared and implanted into the corneal stroma of male BALB/c mice. After 7 days the animals were sacrificed and the corneas were harvested for quantitative analysis of HA and LA.

### Ocular Administration of Compounds

BALB/c mice were randomized to receive RvD1, RvE1 or vehicle (normal saline) by subconjunctival injection in a masked fashion after suture or hydron pellet placement. The compounds were administered at a dose of 100 ng/10 μl per mouse every 48 h after suture or pellet placed. For these experiments, RvD1 and RvE1 were prepared by total organic synthesis as reported previously,^{17,18} in the total organic synthesis core for NIH P-50 DE0169191. The physical properties were monitored routinely via LC/MS/MS matching the reported biological and physical properties prior to analysis in present experiments.

### RNA Isolation and Reverse Transcriptase (RT)-PCR

Corneas were carefully dissected to ensure that the conjunctival and iris tissues were not included. To extract mRNA from whole-thickness corneas, two corneas were pooled as a sample in each group. To extract mRNA from corneal epithelial and stroma-endothelial layers separately, intact corneas were placed in 30 μl of RNA stabilization reagent (RNAlater, Qiagen, Valencia, Calif.) at 4° C. overnight and then stored at −30° C. for 2-3 days. After incubated in 250 μl 20 mM EDTA (sterile, pH 7.4) at 37° C. for 30 minutes, the epithelial layers were peeled off the stroma-endothelial layers before mRNA isolation. Ten corneal epithelial layers or stroma-endothelial layers were pooled as a sample in each group. The mRNA isolated from submandibular lymph nodes were used as positive controls.

A combined-method for total RNA isolation was employed, using Trizol (Invitrogen Corp., Carlsbad, Calif.) and RNeasy MinElute Spin Columns (Qiagen, Valencia, Calif.), as described previously.²¹ Reverse transcription of total RNA was conducted using oligo(dT)₂₀ primer and Superscript™ III Reverse Transcriptase (Invitrogen, Carlsbad, Calif.). PCR was conducted using primer pairs for ChemR23 (sense ACCACACCCTCTACCTGCTG, antisense TGGTGAAGCTCCTGTGACTG, 237 bp) and GAPDH (sense GAAGGGCATCTTGGGCTACAC, antisense GCAGCGAACTTTATTGATGGTATT, 373 bp). The PCR conditions were 35 cycles at 95° C. for 30 seconds, 56° C. for 30 seconds, and 72° C. for 1 minute, followed by finial extension at 72° C. for 10 minutes. PCR products were observed by agarose gel electrophoresis. The mean density of each band was measured by using NIH image J software. The density of each receptor band was divided by the density of the corresponding GAPDH band to obtain the normalized band density.

### Real-Time PCR

1 μl of total cDNA, synthesized from 400 ng total RNA with random hexamers using Superscript™ III Reverse Transcriptase (Invitrogen, Carlsbad, Calif.), was loaded in each well and assays were performed in triplicates. Quantitative PCR was performed with Taqman Universal PCR Mastermix and FAM-MGB dye labeled predesigned primers (Applied Biosystems, Foster City, Calif.) for IL-1α (Mm 99999060_ml), TNF-α (Mm99999068_ml), IL-1β (Mm00434228_ml), VEGFR2 (Mm00440099_ml), VEGFR3 (Mm00433337_ml), VEGFA (Mm00437304_ml), VEGFC (Mm00437313_ml). PCR conditions were 2 minutes at 50° C., 10 minutes at 95° C., followed by 35 cycles of 15 second at 95° C. and 60° C. for 1 minute using an ABI PRISM 7900 HT (Applied Biosystems, CA). PCR amplification of the house-keeping gene encoding GAPDH (Mm999999915_gl) was performed during each run for each sample to allow normalization between samples. A nontemplate control was included in all the experiments to evaluate DNA contamination of isolated RNA and reagents. The results were analyzed by comparative threshold cycle (C_{T}) method. The relative expression level of each sample was expressed as fold change from normal control.

### Isolation of Cornea-infiltrating Cells

Forty corneas were pooled, teased with scissors, and digested with collagenase D (Roche Applied Science, 11088874103) at 37° C. for 1 h in a humidified atmosphere of 5% CO₂. After incubation, corneas were disrupted by grinding with a syringe plunger.²²⁻²⁴ Total cells were then collected after passing through a steel mesh. Upon blockade by anti-FcR mAb, these cells were labeled with FITC-conjugated rat anti-mouse CD11b (granulocyte/monocyte/macrophage marker, BD Pharmingen, San Diego, Calif.) at 4° C. for 30 minutes. CD11b⁺ cells were sorted from total cells by using MoFlo® High-Performance Cell Sorter (Cytomation, Fort Colins, Colo.).

### MK/T-1 Cell Culture and Stimulation

MK/T-1 cells, immortalized keratocytes from the corneal stroma of C57BL/6 mouse (gift from R.L. Gendron [Memorial University of Newfoundland, St. John's, Newfoundland, Canada]), were used to identify the expression of lipid mediator receptors on the corneal keratocytes. MK/T-1 cells were grown in low-glucose Dulbecco's minimum essential medium supplemented with 10% fetal bovine serum and 1 mM α-glutamine at 37° C. in 5% CO₂. To stimulate MK/T-1 cells, 10 ng/μl TNF-α (R&D Systems, Minneapolis, Minn.) and 10 ng/μl IL-1β were added in the culture medium.

### Immunohistochemical Studies

Full thickness corneal tissue or 8 μm-frozen sections were fixed in acetone for 15 minutes at room temperature. To block non-specific staining, anti-FcR mAb (CD16/CD31, FcγIII/II receptor) or 10% goat serum was used before primary antibodies or isotype-matched control antibodies were applied at 4° C. overnight. Thereafter, samples were incubated with secondary antibodies at RT. Each step was followed by three thorough washings in PBS for 5-10 minutes. Finally, the samples were covered with mounting medium (Vector Laboratories, Burlingame, Calif.) and analyzed by epifluorescence microscopy (Eclipse E800; Nikon, Tokyo, Japan). The following antibodies were used: FITC-conjugated rat anti-mouse CD31 (Santa Cruz Biotechnology, Santa Cruz, Calif.), purified rat anti-mouse neutrophil (NIMP-R14, Abcam, Cambridge, Mass.), purified rat anti-mouse F4/80 (Novus Biologicals, Littleton, Colo.) and purified rabbit anti-mouse LYVE-1 (Abcam, Cambridge, Mass.). The secondary antibodies were Rodamine-conjugated donkey anti-rabbit IgG and Rodamine-conjugated goat anti-rat IgG (Santa Cruz Biotechnology, Santa Cruz, Calif.). Isotype controls included FITC-conjugated rat IgG2a, purified rat IgG2b, and purified rabbit IgG (Santa Cruz Biotechnology, Santa Cruz, Calif.).

To quantify corneal angiogenesis, digital pictures of corneal flat-mounts were taken using an image analysis system (Spot Image Analysis, Diagnostic Instruments, Sterling Heights, Mich.). The areas covered by CD31^{high}/LYVE-1⁻-vessels (blood vessels) or CD31^{low}LYVE-1^{high} vessels (lymphatics) were measured morphometrically using NIH Image J software (version 1.34s; http://rsb.info.nih.gov/ij). The total corneal area was outlined using the inner-most vessel of the limbal arcade as the border. The vessel density was calculated by the proportion of neovascularized area to the whole corneal area.

### Statistics

All data are expressed as means±SEM. Statistical significance between the vehicle and each lipid mediator group was analyzed by the two-tailed t-test with Prism (version 4.0; GraphPad, San Diego, Calif.).

Resolvins

The present invention provides methods for preparing novel anti-inflammatory agents and discloses the structures of novel endogenously generated anti-inflammatory mediators that are generated in resolution. The invention is based on the structural elucidation of several new classes of compounds that are generated in vivo during inflammation, which are termed “resolvins.” The structural elucidation of the compounds and the mechanisms of their biosynthesis at sites of inflammation in vivo in murine systems via vascular leukocyte interactions and in brain when aspirin is taken are presented throughout the specification. This structural elucidation of novel biochemical pathways and compounds that serve as endogenous mediators in anti-inflammation and/or pro-resolution forms the basis of a novel approach to active anti-inflammatories that expedites resolution.

From structural elucidation, these novel compounds are “active ingredients” that the body converts via novel biochemical pathways to endogenous omega-3 fatty acid-derived mediators that have anti-inflammatory properties that we've uncovered in murine models. These results provide that these compounds, when generated in vivo in humans, are responsible, at least in part, for the beneficial actions of eating fish and aspirin therapy.

The structural elucidation of these pathways, biological properties and structural elucidation of novel compounds formulates the basis for a novel therapeutic approach, namely administering these compounds and/or related structures/analogs with greater biostability and chemical stability as new therapeutic approaches to expedite resolution and evoke anti-inflammation status.

Along these lines, the new structures, pathways, and examples of novel chemical classes of analogs based on these natural resolvin compounds are presented in the illustrations and figures throughout this specification. Most importantly, with the description of these novel pathways and physical properties of the resolvins, one claim can be directed for assaying these compounds in human fluids (blood, urine, breast milk), biopsied material, etc. as treatment markers to gauge effective n-3 status levels as indices for developing a therapeutic basis for anti-inflammation. This includes LC-MS-MS and GC-MS properties and could also lead to the development of much easier to handle ELISA assays monitoring these novel products.

The phrase “resolvin mediated interaction” is intended to include disease states or conditions caused by or associated with one or more types of inflammation associated with cytokine, leukocyte or PMN regulation and regulation by one or more of the therapeutic analogs described throughout the specification for the pharmacologic inhibition of NV, hemangiogenesis and/or angiogenic condition(s) of the cornea. In one embodiment, the disease state includes, for example, those diseases that afflict a subject by associating with or interfering with cytokine, leukocyte or PMN regulation within the subject. Such disease states or conditions are described throughout the specification, vide infra, and are incorporated herein in their entirety. Presently unknown conditions related to cytokine, leukocyte or PMN regulation that may be discovered in the future are encompassed by the present invention, since the characterization as conditions related to resolvin mediated interaction(s) will be readily determinable by persons skilled in the art.

Resolvins are natural counter regulatory lipid mediators in host defense mechanisms that protect host tissues from effector cell mediated injury and over amplification of acute inflammation to dampen the inflammatory response, i.e., counterregulative. Some known chronic inflammatory diseases may represent the loss of and/or genetically program low resolvin endogenous responders and/or levels. The resolvin analogs described throughout the specification can be used to replace, enhance and/or treat the loss of these substances therapeutically and thereby pharmacologically resolve inflammation by inhibiting leukocyte recruitment and amplification, namely inhibition of the amplification of inflammation.

The present invention is also drawn to methods for treating disease states or conditions that are associated with inflammation (hence “resolving”), the recruitment of neutrophils, leukocytes and/or cytokines are included within the general scope of NV, hemangiogenesis and/or angiogenic condition(s) of the cornea.

Given the cellular origins of the different chambers of the eye, it is not possible to assume that the same targets are present in the front or cornea of the eye versus the retina or any other part of the eye; each need to be established separately both in experimental laboratory settings in animals and with the rigor of clinical trials in human studies.

The eye, being a highly specialized sensory organ, has cells in the anterior chamber that differ in their origin from cells in the posterior chamber. Therefore, therapeutic approaches and current treatments of the cornea vs. retinal layer and sclera retinal-glial scleral layer are not and cannot be assumed to be the same.

Because of the anatomical structural and functional differences in each section of the eye, therapeutics need to be targeted to precise locations within the eye, for example, cornea vs. retina, in order to achieve appropriate therapeutic interventions. The cell types and their pathologies in these sections of the human eye are different.

A point of fact that the cornea versus the retina are physiologically of a different ilk has given rise to very different study groups at the national level as currently considered by the NIH. For example, the anterior portion of the eye and associated structures, e.g. the cornea, canal of Schlemm, lens, etc. are addressed by a specific study section at the level of the NIH, whereas structures of the posterior chamber and section of the eye including retina, optic nerve, etc. are addressed by a separate study section. This is because the tissue and cell types from the posterior of the eye are largely of neural origin or highly specialized non-photosensitive epithelial layers, such as in the retina, whereas cells in the cornea are nonvascular, non-refractive epithelial cells. It is these cell type differences and anatomical differences that give rise to different treatment distinctions.

Agents that act on cells of the human retina (anti-VEGF therapies, etc.) do not have specific targeted actions within the healthy human cornea per se and current therapies along these lines need to be injected within the vitreous body posterior chamber of the eye. These treatments include, for example, anti-VEGF therapies.

The present surprisingly demonstrates the unique suture model of angiogenesis in the cornea and resolvins are able to protect from neovascularization of corneal tissues. Without this experimental evidence put forth in the specification, this could not be anticipated or was predictable.

More importantly, limited therapeutics are available to topically treat inflammation in the cornea that are also able to regulate unwanted neovascularization of the corneal tissue. Current anti-inflammatories for topical treatments in the eye, i.e., applied directly to the cornea, include steroids, which are well appreciated by the clinical community to have long-term deleterious side effects.

The current findings that specific resolvins and protectins are each anti-inflammatory topically as well as prevent neovascularization in a cornea suture model is an ideal example of novel dual-pronged actions of the resolvins and protectins in cornea. In the cornea, neovascularization is unwanted as it can limit vision.

Corneal replacements are routinely carried out and can be associated in humans with suture-induced/initiated local inflammation. Topical application of resolvins or protectins as demonstrated for the first time in direct comparison from the present results could be useful in the treatment of this currently unmet clinical need.

“Alkyl” by itself or as part of another substituent refers to a saturated or unsaturated branched, straight-chain or cyclic monovalent hydrocarbon radical having the stated number of carbon atoms (i.e., C1-C6 means one to six carbon atoms) that is derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl, cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. Where specific levels of saturation are intended, the nomenclature “alkanyl,” “alkenyl” and/or “alkynyl” is used, as defined below. In preferred embodiments, the alkyl groups are (C1-C6) alkyl.

“Alkanyl” by itself or as part of another substituent refers to a saturated branched, straight-chain or cyclic alkyl derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like. In preferred embodiments, the alkanyl groups are (C1-C6) alkanyl.

“Alkenyl” by itself or as part of another substituent refers to an unsaturated branched, straight-chain or cyclic alkyl having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like. In preferred embodiments, the alkenyl group is (C2-C6) alkenyl.

“Alkynyl” by itself or as part of another substituent refers to an unsaturated branched, straight-chain or cyclic alkyl having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like. In preferred embodiments, the alkynyl group is (C2-C6) alkynyl.

“Alkyldiyl” by itself or as part of another substituent refers to a saturated or unsaturated, branched, straight-chain or cyclic divalent hydrocarbon group having the stated number of carbon atoms (i.e., C1-C6 means from one to six carbon atoms) derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyl groups include, but are not limited to, methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkanyldiyl, alkenyldiyl and/or alkynyldiyl is used. Where it is specifically intended that the two valencies are on the same carbon atom, the nomenclature “alkylidene” is used. In preferred embodiments, the alkyldiyl group is (C1-C6) alkyldiyl. Also preferred are saturated acyclic alkanyldiyl groups in which the radical centers are at the terminal carbons, e.g., methandiyl (methano); ethan-1,2-diyl(ethano); propan-1,3-diyl (propano); butan-1,4-diyl (butano); and the like (also referred to as alkylenos, defined infra).

“Alkyleno” by itself or as part of another substituent refers to a straight-chain saturated or unsaturated alkyldiyl group having two terminal monovalent radical centers derived by the removal of one hydrogen atom from each of the two terminal carbon atoms of straight-chain parent alkane, alkene or alkyne. The locant of a double bond or triple bond, if present, in a particular alkyleno is indicated in square brackets. Typical alkyleno groups include, but are not limited to, methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, buta[1,3]diyno, etc.; and the like. Where specific levels of saturation are intended, the nomenclature alkano, alkeno and/or alkyno is used. In preferred embodiments, the alkyleno group is (C1-C6) or (C1-C3) alkyleno. Also preferred are straight-chain saturated alkano groups, e.g., methano, ethano, propano, butano, and the like.

“Heteroalkyl,” Heteroalkanyl,” Heteroalkenyl,” Heteroalkynyl,” Heteroalkyldiyl” and “Heteroalkyleno” by themselves or as part of another substituent refer to alkyl, alkanyl, alkenyl, alkynyl, alkyldiyl and alkyleno groups, respectively, in which one or more of the carbon atoms are each independently replaced with the same or different heteratoms or heteroatomic groups. Typical heteroatoms and/or heteroatomic groups which can replace the carbon atoms include, but are not limited to, —O—, —S—, —S—O—, —NR′—, —PH—, —S(O)—, —S(O)₂—, —S(O)NR′—, —S(O)₂NR′—, and the like, including combinations thereof, where each R′ is independently hydrogen or (C1-C6) alkyl.

“Cycloalkyl” and “Heterocycloalkyl” by themselves or as part of another substituent refer to cyclic versions of “alkyl” and “heteroalkyl” groups, respectively. For heteroalkyl groups, a heteroatom can occupy the position that is attached to the remainder of the molecule. Typical cycloalkyl groups include, but are not limited to, cyclopropyl; cyclobutyls such as cyclobutanyl and cyclobutenyl; cyclopentyls such as cyclopentanyl and cyclopentenyl; cyclohexyls such as cyclohexanyl and cyclohexenyl; and the like. Typical heterocycloalkyl groups include, but are not limited to, tetrahydrofuranyl (e.g., tetrahydrofuran-2-yl, tetrahydrofuran-3-yl, etc.), piperidinyl (e.g., piperidin-1-yl, piperidin-2-yl, etc.), morpholinyl (e.g., morpholin-3-yl, morpholin-4-yl, etc.), piperazinyl (e.g., piperazin-1-yl, piperazin-2-yl, etc.), and the like.

“Acyclic Heteroatomic Bridge” refers to a divalent bridge in which the backbone atoms are exclusively heteroatoms and/or heteroatomic groups. Typical acyclic heteroatomic bridges include, but are not limited to, —O—, —S—, —S—O—, —NR′—, —PH—, —S(O)—, —S(O)₂—, —S(O)NR′—, —S(O)₂NR′—, and the like, including combinations thereof, where each R′ is independently hydrogen or (C1-C6) alkyl.

“Parent Aromatic Ring System” refers to an unsaturated cyclic or polycyclic ring system having a conjugated π electron system. Specifically included within the definition of “parent aromatic ring system” are fused ring systems in which one or more of the rings are aromatic and one or more of the rings are saturated or unsaturated, such as, for example, fluorene, indane, indene, phenalene, tetrahydronaphthalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, tetrahydronaphthalene, triphenylene, trinaphthalene, and the like, as well as the various hydro isomers thereof.

“Aryl” by itself or as part of another substituent refers to a monovalent aromatic hydrocarbon group having the stated number of carbon atoms (i.e., C5-C15 means from 5 to 15 carbon atoms) derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like, as well as the various hydro isomers thereof. In preferred embodiments, the aryl group is (C5-C15) aryl, with (C5-C10) being even more preferred. Particularly preferred aryls are cyclopentadienyl, phenyl and naphthyl.

“Arylaryl” by itself or as part of another substituent refers to a monovalent hydrocarbon group derived by the removal of one hydrogen atom from a single carbon atom of a ring system in which two or more identical or non-identical parent aromatic ring systems are joined directly together by a single bond, where the number of such direct ring junctions is one less than the number of parent aromatic ring systems involved. Typical arylaryl groups include, but are not limited to, biphenyl, triphenyl, phenyl-naphthyl, binaphthyl, biphenyl-naphthyl, and the like. Where the number of carbon atoms in an arylaryl group are specified, the numbers refer to the carbon atoms comprising each parent aromatic ring. For example, (C5-C15) arylaryl is an arylaryl group in which each aromatic ring comprises from 5 to 15 carbons, e.g., biphenyl, triphenyl, binaphthyl, phenylnaphthyl, etc. Preferably, each parent aromatic ring system of an arylaryl group is independently a (C5-C15) aromatic, more preferably a (C5-C10) aromatic. Also preferred are arylaryl groups in which all of the parent aromatic ring systems are identical, e.g., biphenyl, triphenyl, binaphthyl, trinaphthyl, etc.

“Biaryl” by itself or as part of another substituent refers to an arylaryl group having two identical parent aromatic systems joined directly together by a single bond. Typical biaryl groups include, but are not limited to, biphenyl, binaphthyl, bianthracyl, and the like. Preferably, the aromatic ring systems are (C5-C15) aromatic rings, more preferably (C5-C10) aromatic rings. A particularly preferred biaryl group is biphenyl.

“Arylalkyl” by itself or as part of another substituent refers to an acyclic alkyl group in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylakenyl and/or arylalkynyl is used. In preferred embodiments, the arylalkyl group is (C6-C21) arylalkyl, e.g., the alkanyl, alkenyl or alkynyl moiety of the arylalkyl group is (C1-C6) and the aryl moiety is (C5-C15). In particularly preferred embodiments the arylalkyl group is (C6-C13), e.g., the alkanyl, alkenyl or alkynyl moiety of the arylalkyl group is (C1-C3) and the aryl moiety is (C5-C10).

“Parent Heteroaromatic Ring System” refers to a parent aromatic ring system in which one or more carbon atoms are each independently replaced with the same or different heteroatoms or heteroatomic groups. Typical heteroatoms or heteroatomic groups to replace the carbon atoms include, but are not limited to, N, NH, P, O, S, S(O), S(O)₂, Si, etc. Specifically included within the definition of “parent heteroaromatic ring systems” are fused ring systems in which one or more of the rings are aromatic and one or more of the rings are saturated or unsaturated, such as, for example, benzodioxan, benzofuran, chromane, chromene, indole, indoline, xanthene, etc. Also included in the definition of “parent heteroaromatic ring system” are those recognized rings that include common substituents, such as, for example, benzopyrone and 1-methyl-1,2,3,4-tetrazole. Typical parent heteroaromatic ring systems include, but are not limited to, acridine, benzimidazole, benzisoxazole, benzodioxan, benzodioxole, benzofuran, benzopyrone, benzothiadiazole, benzothiazole, benzotriazole, benzoxaxine, benzoxazole, benzoxazoline, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Heteroaryl” by itself or as part of another substituent refers to a monovalent heteroaromatic group having the stated number of ring atoms (e.g., “5-14 membered” means from 5 to 14 ring atoms) derived by the removal of one hydrogen atom from a single atom of a parent heteroaromatic ring system. Typical heteroaryl groups include, but are not limited to, groups derived from acridine, benzimidazole, benzisoxazole, benzodioxan, benzodiaxole, benzofuran, benzopyrone, benzothiadiazole, benzothiazole, benzotriazole, benzoxazine, benzoxazole, benzoxazoline, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like, as well as the various hydro isomers thereof. In preferred embodiments, the heteroaryl group is a 5-14 membered heteroaryl, with 5-10 membered heteroaryl being particularly preferred.

“Heteroaryl-Heteroaryl” by itself or as part of another substituent refers to a monovalent heteroaromatic group derived by the removal of one hydrogen atom from a single atom of a ring system in which two or more identical or non-identical parent heteroaromatic ring systems are joined directly together by a single bond, where the number of such direct ring junctions is one less than the number of parent heteroaromatic ring systems involved. Typical heteroaryl-heteroaryl groups include, but are not limited to, bipyridyl, tripyridyl, pyridylpurinyl, bipurinyl, etc. Where the number of atoms are specified, the numbers refer to the number of atoms comprising each parent heteroaromatic ring systems. For example, 5-15 membered heteroaryl-heteroaryl is a heteroaryl-heteroaryl group in which each parent heteroaromatic ring system comprises from 5 to 15 atoms, e.g., bipyridyl, tripuridyl, etc. Preferably, each parent heteroaromatic ring system is independently a 5-15 membered heteroaromatic, more preferably a 5-10 membered heteroaromatic. Also preferred are heteroaryl-heteroaryl groups in which all of the parent heteroaromatic ring systems are identical.

“Biheteroaryl” by itself or as part of another substituent refers to a heteroaryl-heteroaryl group having two identical parent heteroaromatic ring systems joined directly together by a single bond. Typical biheteroaryl groups include, but are not limited to, bipyridyl, bipurinyl, biquinolinyl, and the like. Preferably, the heteroaromatic ring systems are 5-15 membered heteroaromatic rings, more preferably 5-10 membered heteroaromatic rings.

“Heteroarylalkyl” by itself or as part of another substituent refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylakenyl and/or heteroarylalkynyl is used. In preferred embodiments, the heteroarylalkyl group is a 6-21 membered heteroarylalkyl, e.g., the alkanyl, alkenyl or alkynyl moiety of the heteroarylalkyl is (C1-C6) alkyl and the heteroaryl moiety is a 5-15-membered heteroaryl. In particularly preferred embodiments, the heteroarylalkyl is a 6-13 membered heteroarylalkyl, e.g., the alkanyl, alkenyl or alkynyl moiety is (C1-C3) alkyl and the heteroaryl moiety is a 5-10 membered heteroaryl.

“Halogen” or “Halo” by themselves or as part of another substituent, unless otherwise stated, refer to fluoro, chloro, bromo and iodo.

“Haloalkyl” by itself or as part of another substituent refers to an alkyl group in which one or more of the hydrogen atoms is replaced with a halogen. Thus, the term “haloalkyl” is meant to include monohaloalkyls, dihaloalkyls, trihaloalkyls, etc. up to perhaloalkyls. For example, the expression “(C1-C2) haloalkyl” includes fluoromethyl, difluoromethyl, trifluoromethyl, 1-fluoroethyl, 1,1-difluoroethyl, 1,2-difluoroethyl, 1,1,1-trifluoroethyl, perfluoroethyl, etc.

The above-defined groups may include prefixes and/or suffixes that are commonly used in the art to create additional well-recognized substituent groups. As examples, “alkyloxy” or “alkoxy” refers to a group of the formula —OR″, “alkylamine” refers to a group of the formula —NHR″ and “dialkylamine” refers to a group of the formula —NR″R″, where each R″ is independently an alkyl. As another example, “haloalkoxy” or “haloalkyloxy” refers to a group of the formula —OR′″, where R′″ is a haloalkyl.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxylprotecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPPS groups), glycol ethers, such as ethylene glycol and propylene glycol derivatives and allyl ethers.

Throughout the following descriptions, it should be understood that where particular double bonding is depicted, it is intended to include both cis and trans configurations. Exemplary formulae are provided with specific configurations, but for completeness, the double bonds can be varied. Not every structural isomer is shown in efforts to maintain brevity of the specification. However, this should not be considered limiting in nature. Additionally, where synthetic schemes are provided, it should be understood that all cis/trans configurational isomers are also contemplated and are within the scope and purview of the synthesis. Again, particular double bonding is depicted in exemplary manner.

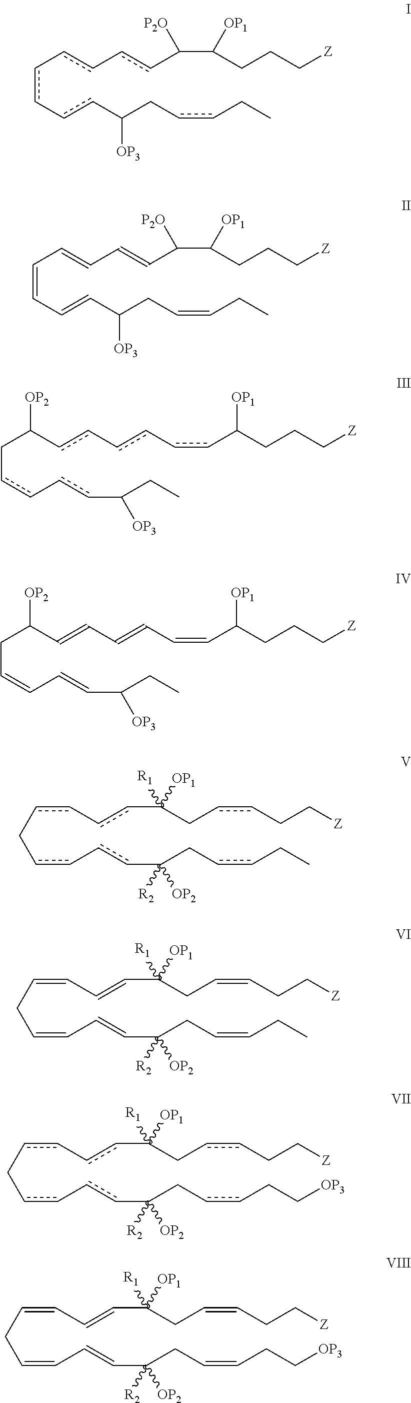

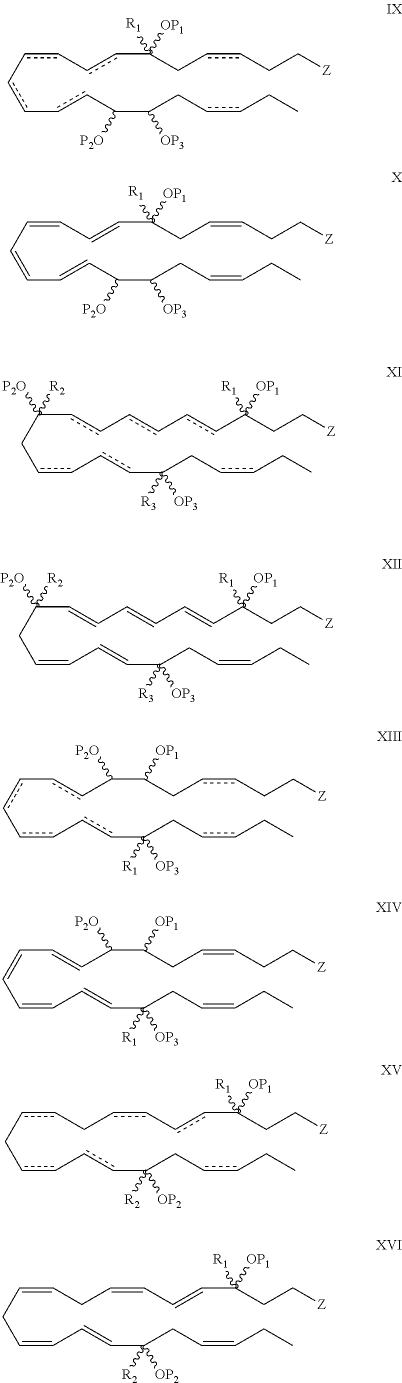

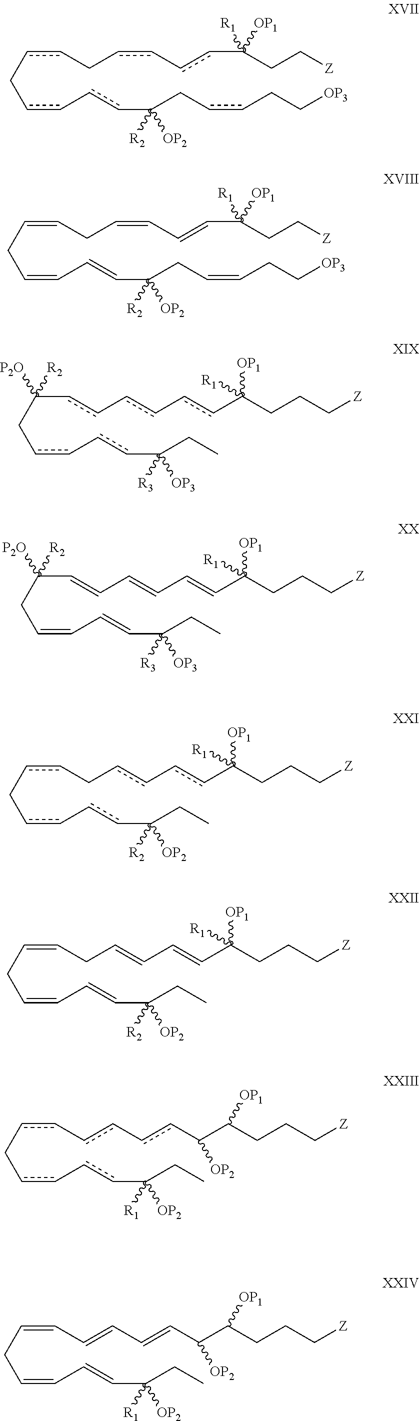

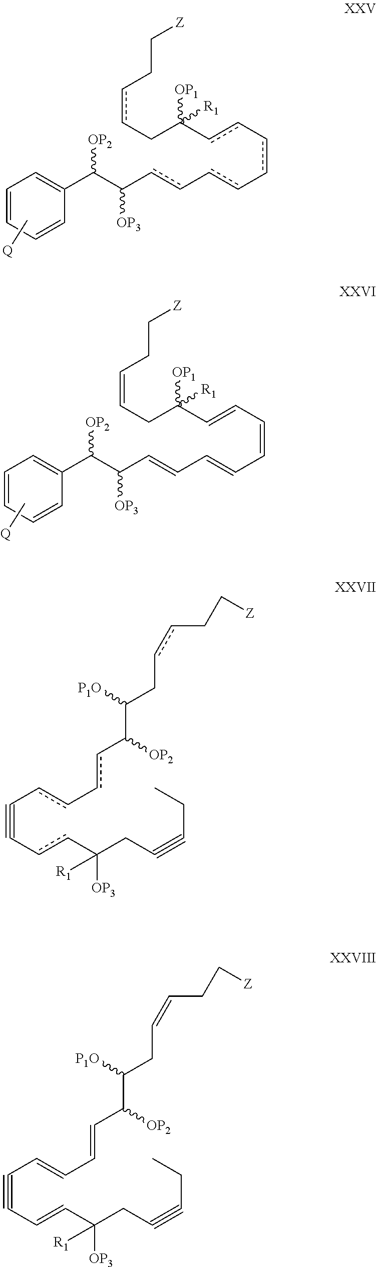

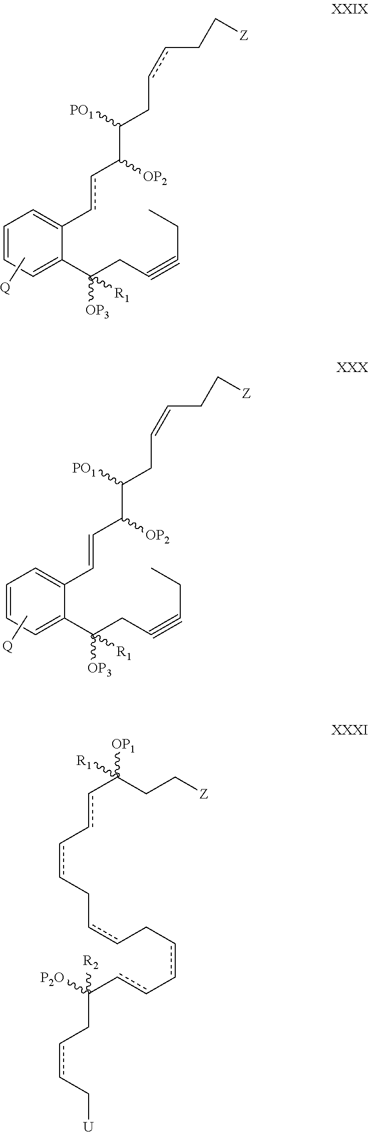

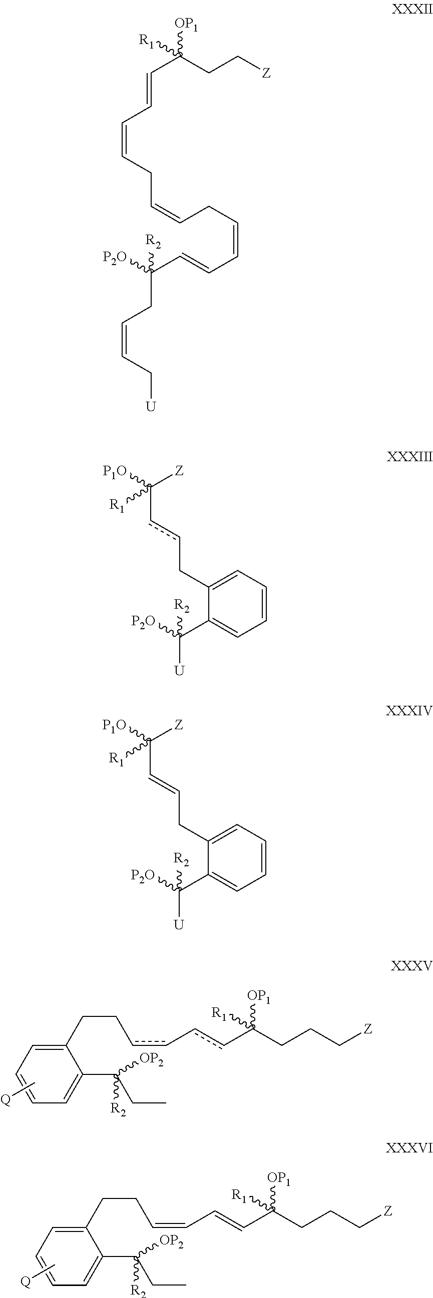

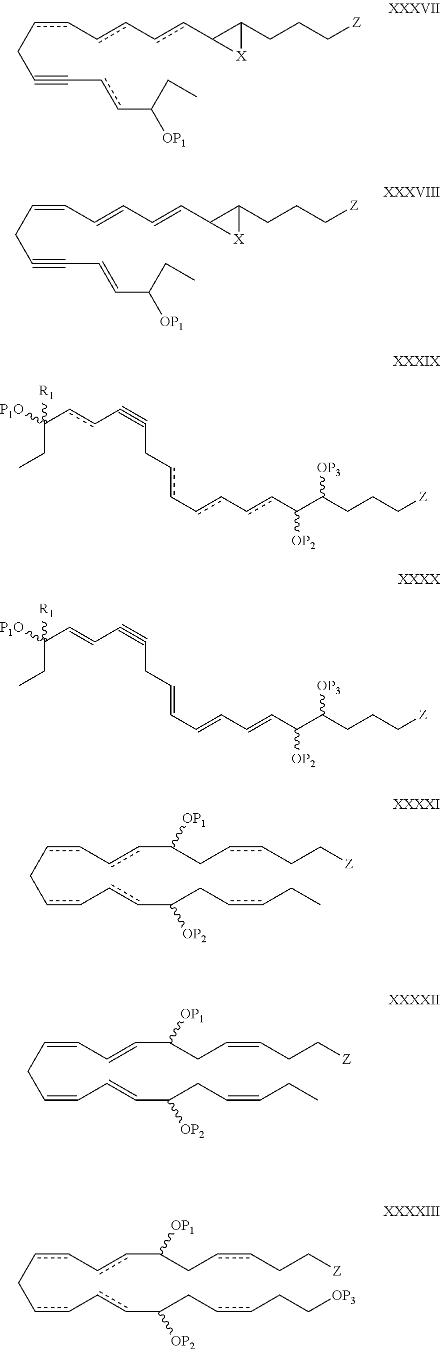

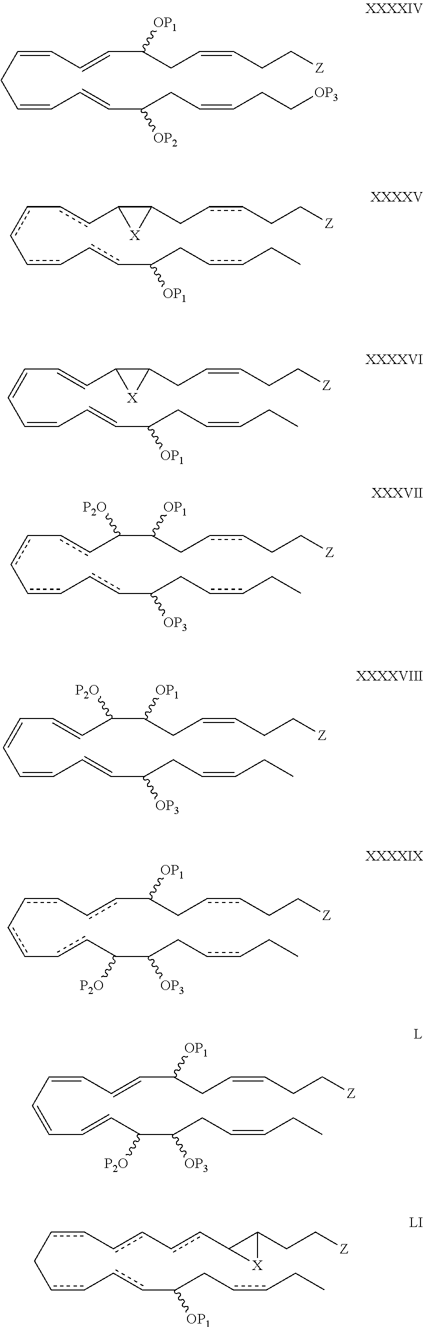

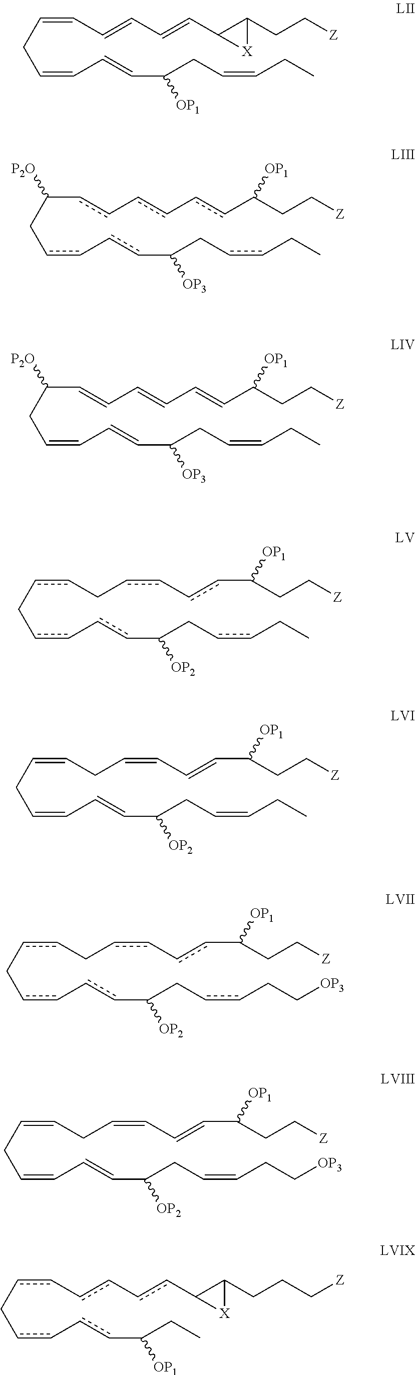

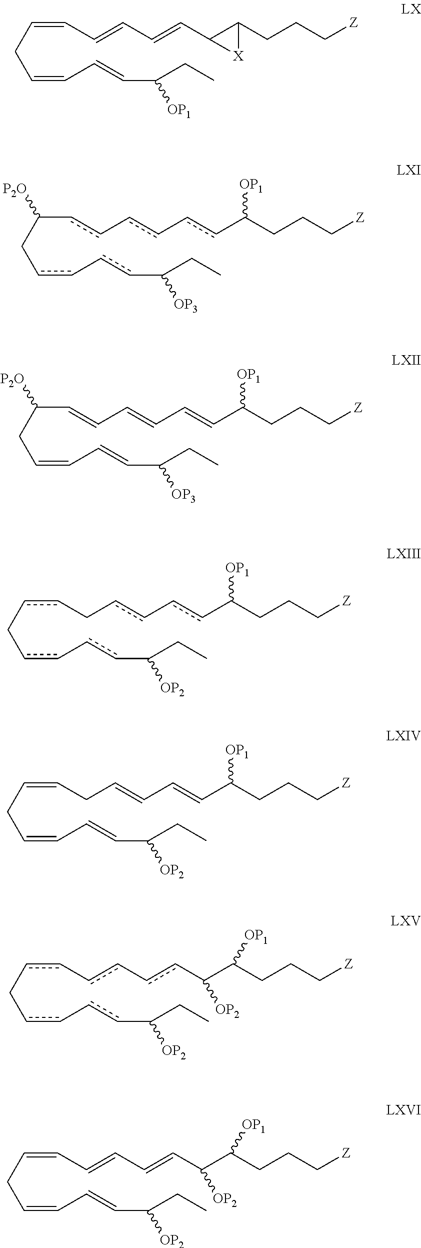

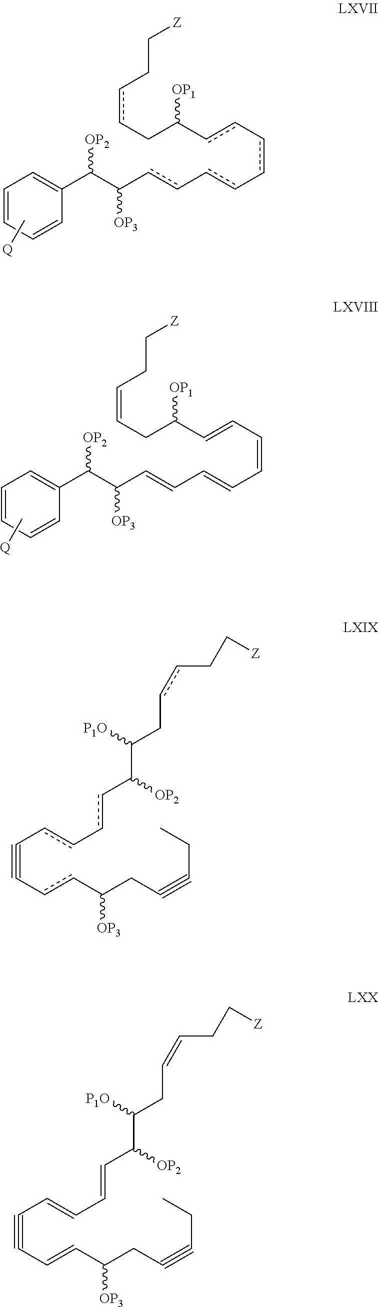

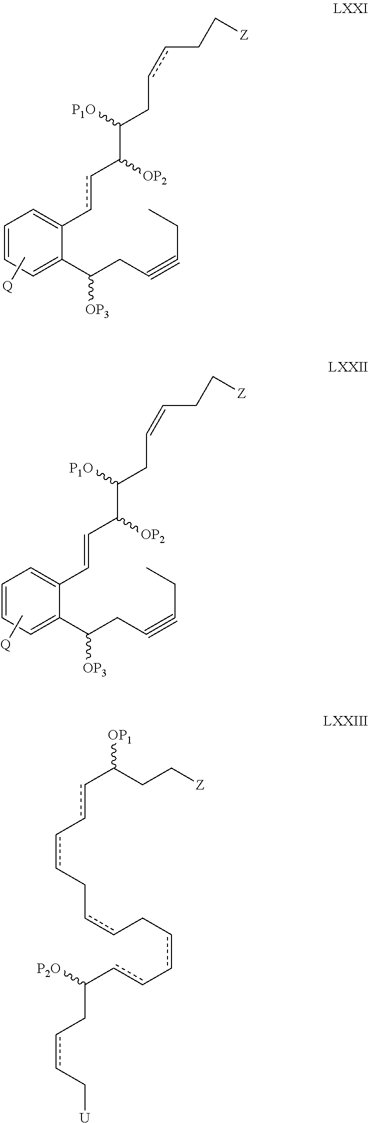

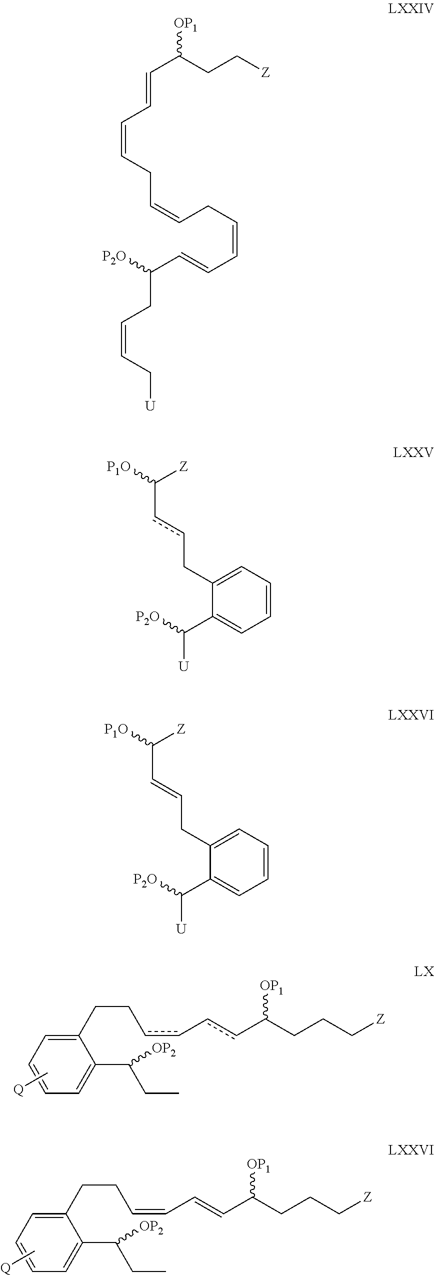

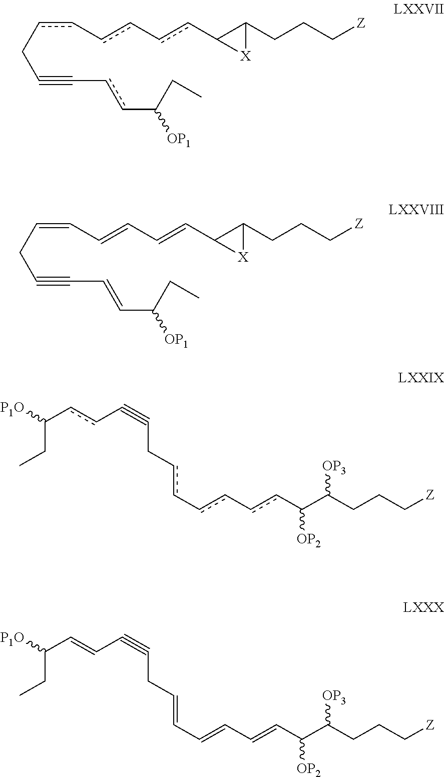

wherein a bond depicted as [symbol] represents either a cis or trans double bond;

wherein P₁, P₂ and P₃, if present, each individually are protecting groups, hydrogen atoms or combinations thereof;

wherein R₁, R₂ and R₃, if present, each individually are substituted or unsubstituted, branched or unbranched alkyl groups, substituted or unsubstituted aryl groups, substituted or unsubstituted, branched or unbranched alkylaryl groups, halogen atoms, hydrogen atoms or combinations thereof;

wherein Z is —C(O)OR^{d}, —C(O)NR^{c}R^{c}, —C(O)H, —C(NH)NR^{c}R^{c}, —C(S)H, —C(S)OR^{d}, —C(S)NR^{c}R^{c}, —CN;

each R^{a}, if present, is independently selected from the group consisting of hydrogen, (C1-C6) alkyl, (C3-C8) cycloalkyl, cyclohexyl, (C4-C11) cycloalkylalkyl, (C5-C10) aryl, phenyl, (C6-C16) arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;

each R^{b}, if present, is a suitable group independently selected from the group consisting of ═O, —OR^{d}, (C1-C3) haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};

each R^{c}, if present, is independently a protecting group or R^{a}, or, alternatively, each R^{c} is taken together with the nitrogen atom to which it is bonded to form a 5 to 8-membered cycloheteroalkyl or heteroaryl which may optionally include one or more of the same or different additional heteroatoms and which may optionally be substituted with one or more of the same or different R^{a} or suitable R^{b} groups;

each n, independently, if present, is an integer from 0 to 3;

each R^{d}, independently, if present, is a protecting group or R^{a};

in particular, Z is a carboxylic acid, ester, amide, thiocarbamate, carbamate, thioester, thiocarboxamide or a nitrile;

wherein X, if present, is a substituted or unsubstituted methylene, an oxygen atom, a substituted or unsubstituted nitrogen atom, or a sulfur atom;

wherein Q, if present, represents one or more substituents and each Q individually, if present, is a halogen atom or a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, amino, hydroxy, cyano, carboxyl, alkoxycarbonyloxy, aryloxycarbonyloxy or aminocarbonyl group;

wherein U, if present, is a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, alkoxycarbonyloxy, and aryloxycarbonyloxy group;

and pharmaceutically acceptable salts thereof.

In certain embodiments, Z is a pharmaceutically acceptable salt of a carboxylic acid, and in particular is an ammonium salt or forms a prodrug.

In certain embodiments, P₁, P₂, and P₃, if present, each individually are hydrogen atoms and Z is a carboxylic acid or ester. In other embodiments, X is an oxygen atom, one or more P's are hydrogen atoms, and Z is a carboxylic acid or ester. In still other embodiments, Q is one or more halogen atoms, one or more P's are hydrogen atoms, and Z is a carboxylic acid or ester.

In certain embodiments, R₁, R₂ and R₃, if present, are each individually lower alkyl groups, such as methyl, ethyl, and propyl and can be halogenated, such as trifluoromethyl. In one aspect, at least one of R₁, R₂ and R₃, if present, is not a hydrogen atom. Generally, Z is a carboxylic acid and one or more P's are hydrogen atoms.

In certain embodiments, when OP₃ is disposed terminally within the resolvin analog, the protecting group can be removed to afford a hydroxyl. Alternatively, in certain embodiments, the designation of OP₃ serves to denote that the terminal carbon is substituted with one or more halogens, i.e., the terminal C-18, C-20, or C-22 carbon, is a trifluoromethyl group, or arylated with an aryl group that can be substituted or unsubstituted as described herein. Such manipulation at the terminal carbon serves to protect the resolvin analog from omega P₄₅₀ metabolism that can lead to biochemical inactivation.

In certain embodiments, P₁, P₂, and P₃, if present, each individually are hydrogen atoms and Z is a carboxylic ester. In other embodiments, P₁, P₂, and P₃, if present, each individually are hydrogen atoms and Z is not carboxylic acid.

In one aspect, the compounds described herein are isolated and/or purified, in particular, compounds in which P₁, P₂, and P₃, if present, each individually are hydrogen atoms and Z is a carboxylic acid, are isolated and or purified.

In certain aspects of the invention, particular compounds are not included; these include the even numbered compounds identified above by Roman numbers, i.e., II, IV, VI, VIII, X, etc. through LXXX.

The present invention provides compounds and pharmaceutical compositions useful for the treatment of NV, hemangiogenesis and/or angiogenic condition(s) of the cornea, having the formula:

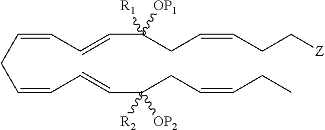

P₁ and P₂ each individually are protecting groups, hydrogen atoms or combinations thereof.

R₁ and R₂ each individually are substituted or unsubstituted, branched or unbranched alkyl groups, substituted or unsubstituted aryl groups, substituted or unsubstituted, branched or unbranched alkylaryl groups, halogen atoms, hydrogen atoms or combinations thereof.

Z is —C(O)OR^{d}, —C(O)NR^{c}R^{c}, —C(O)H, —C(NH)NR^{c}R^{c}, —C(S)H, —C(S)OR^{d}, —C(S)NR^{c}R^{c}, —CN;

each R^{a}, if present, is independently selected from the group consisting of hydrogen, (C1-C6) alkyl, (C3-C8) cycloalkyl, cyclohexyl, (C4-C11) cycloalkylalkyl, (C5-C10) aryl, phenyl, (C6-C16) arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;

each R^{b}, if present, is a suitable group independently selected from the group consisting of ═O, —OR^{d}, (C1-C3) haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]—R^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR)]ₙNR^{c}R^{c};

each R^{c}, if present, is independently a protecting group or R^{a}, or, alternatively, each R^{c} is taken together with the nitrogen atom to which it is bonded to form a 5 to 8-membered cycloheteroalkyl or heteroaryl which may optionally include one or more of the same or different additional heteroatoms and which may optionally be substituted with one or more of the same or different R^{a} or suitable R^{b} groups;

each n, independently, if present, is an integer from 0 to 3;

each R^{d}, independently, if present, is a protecting group or R^{a};

and pharmaceutically acceptable salts thereof.

In certain embodiments, P₁ and P₂ are hydrogen atoms, R₁ and R₂ each individually are methyl groups or hydrogen atoms or combinations thereof, and Z is carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention provides compounds and pharmaceutical compositions useful for the treatment of NV, hemangiogenesis and/or angiogenic condition(s) of the cornea, having the formula:

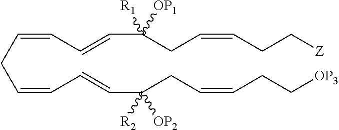

P₁, P₂ and P₃ each individually are protecting groups, hydrogen atoms or combinations thereof and R₁, R₂ and Z are as defined above.

In certain embodiments, P₁, P₂ and P₃ each are hydrogen atoms, R₁ and R₂ each individually are methyl groups or hydrogen atoms or combinations thereof and Z is a carboxylic acid or a carboxylic ester.

In certain aspects the designation of OP₃ serves to denote that the terminal carbon is substituted with one or more halogens (I, Cl, F, Br, mono, di or tri substitution) to form, for example, a trifluoromethyl group, or is an aryl group or phenoxy group that can be substituted or unsubstituted as described herein.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention provides compounds and pharmaceutical compositions useful for the treatment of NV, hemangiogenesis and/or angiogenic condition(s) of the cornea, having the formula:

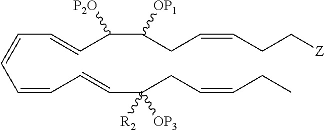

P₁, P₂, P₃, R₁ and Z are as defined above.

In an embodiment, P₁, P₂ and P₃ each are hydrogen atoms, R₁ is a methyl group or a hydrogen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention provides compounds and pharmaceutical compositions useful for the treatment of NV, hemangiogenesis and/or angiogenic condition(s) of the cornea, having the formula:

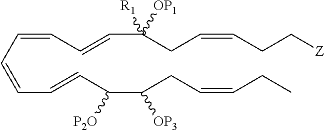

P₁, P₂, P₃, R₁ and Z are as defined above.

In a particular embodiment, P₁, P₂ and P₃ each are hydrogen atoms, R₁ is a methyl group or a hydrogen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention provides compounds and pharmaceutical compositions useful for the treatment of NV, hemangiogenesis and/or angiogenic condition(s) of the corneas, having the formula:

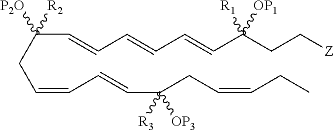

P₁, P₂, P₃ and Z are as defined above.

R₁, R₂ and R₃, each individually are substituted or unsubstituted, branched or unbranched alkyl groups, substituted or unsubstituted aryl groups, substituted or unsubstituted, branched or unbranched alkylaryl groups, halogen atoms, hydrogen atoms or combinations thereof.

In a particular embodiment, P₁, P₂ and P₃ each are hydrogen atoms, R₁, R₂ and R₃ are each hydrogen atoms and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention also provides compounds and pharmaceutical compositions useful for the treatment of NV, hemangiogenesis and/or angiogenic conditions, having the formula:

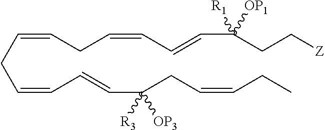

P₁, P₂, R₁, R₂ and Z are as defined above.

In certain embodiments, P₁ and P₂ are hydrogen atoms, R₁ and R₂ each individually are methyl groups or hydrogen atoms or combinations thereof and Z is carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

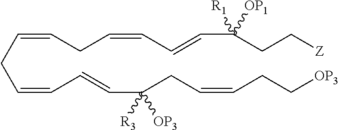

P₁, P₂, P₃, R₁, R₂ and Z are as defined above.

In a particular embodiment, P₁, P₂ and P₃ each are hydrogen atoms and Z is a carboxylic acid or a carboxylic ester.

In certain aspects the designation of OP₃ serves to denote that the terminal carbon is substituted with one or more halogens (I, Cl, F, Br, mono, di or tri substitution) to form, for example, a trifluoromethyl group, or is an aryl group or phenoxy group that can be substituted or unsubstituted as described herein.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention provides compounds and pharmaceutical compositions useful for the treatment of NV, hemangiogenesis and/or angiogenic conditions, having the formula:

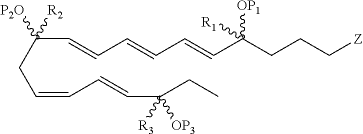

P₁, P₂, P₃, R₁, R₂, R₃ and Z are as defined above.

In one embodiment, P₁, P₂ and P₃ each are hydrogen atoms, R₁, R₂ and R₃ each individually are methyl groups or hydrogen atoms or combinations thereof and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

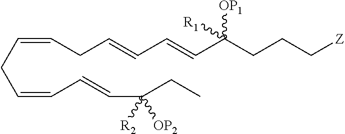

P₁, P₂, R₁, R₂ and are as defined above.

In one particular embodiment, P₁ and P₂ are hydrogen atoms, R₁ and R₂ each individually are methyl groups or hydrogen atoms or combinations thereof and Z is carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

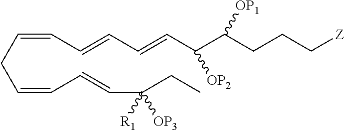

P₁, P₂, P₃, R₁ and Z are as defined above.

In one embodiment, P₁, P₂ and P₃ each are hydrogen atoms, R₁ is a methyl group or a hydrogen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

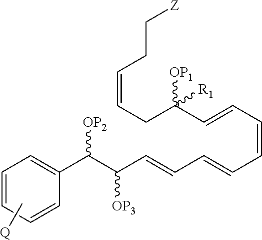

P₁, P₂, P₃, R₁ and Z are as defined above.

Q represents one or more substituents and each Q, independently, is a hydrogen atom, a halogen atom or a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, amino, hydroxy, cyano, carboxyl, alkoxycarbonyloxy, aryloxycarbonyloxy or aminocarbonyl group.

In one particular aspect, P₁, P₂ and P₃ each are hydrogen atoms, R₁ is a methyl group or a hydrogen atom, each Q is a hydrogen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

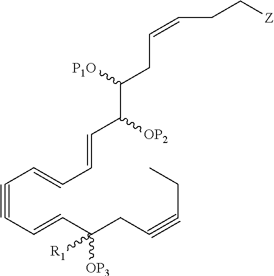

P₁, P₂, P₃, R₁ and Z are as defined above.

In one embodiment, P₁, P₂ and P₃ each are hydrogen atoms, R₁ is a methyl group or a hydrogen atom, and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention also provides compounds and pharmaceutical compositions useful for the treatment of NV, hemangiogenesis and/or angiogenic conditions, having the formula:

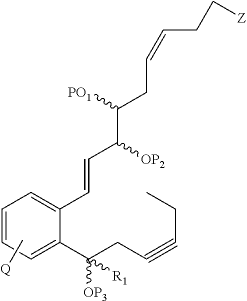

P₁, P₂, P₃, R₁, Q and Z are as defined above.

In one particular embodiment, P₁, P₂ and P₃ each are hydrogen atoms, R is a methyl group or a hydrogen atom, each Q is a hydrogen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

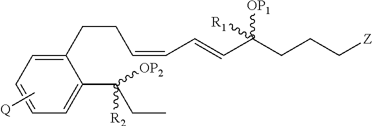

P₁, P₂, R₁, R₂, Q and Z are as defined above.

In one embodiment, P₁, and P₂ each are hydrogen atoms, R₁ and R₂ each individually are methyl groups or hydrogen atoms or combinations thereof, each Q is a hydrogen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

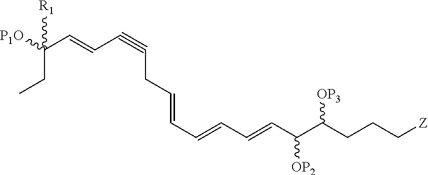

P₁, P₂, P₃, R₁ and Z are as defined above.

In one embodiment, P₁, P₂ and P₃ each are hydrogen atoms, R₁ is a methyl group or a hydrogen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

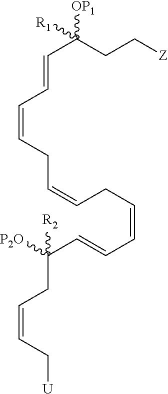

P₁, P₂, R₁, R₂ and Z are as defined above.

U is a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, alkoxycarbonyloxy, and aryloxycarbonyloxy group.

In one aspect, P₁, and P₂ each are hydrogen atoms, R₁ and R₂ each individually are methyl groups or hydrogen atoms or combinations thereof, U is a trifluoromethyl group and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention still further provides compounds and pharmaceutical compositions useful for the treatment of NV, hemangiogenesis and/or angiogenic conditions, having the formula:

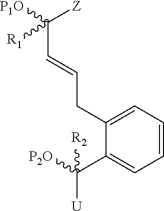

P₁, P₂, R₁, R₂, U and Z are as defined above.

For example, P₁, and P₂ each are hydrogen atoms, R₁ and R₂ each individually are methyl groups or hydrogen atoms or combinations thereof, U is a trifluoromethyl group and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention provides compounds and pharmaceutical compositions useful for the treatment of NV, hemangiogenesis and/or angiogenic conditions, having the formula:

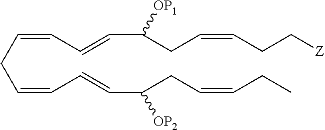

P₁, P₂ and Z are as defined above.

In certain embodiments, P₁ and P₂ are hydrogen atoms and Z is carboxylic acid or a carboxylic ester. In certain embodiments, when P₁ and P₂ are hydrogen atoms and Z is a carboxylic acid, the compound is either isolated and/or purified.

Purity of compounds noted throughout the specification is at least 80% pure, in particular, greater than 90% pure, more particularly greater than 95% pure and most particularly greater than about 99% pure, based on analytical measurements such has GC, MS, or ¹HNMR, etc. This applies to all compounds noted herein, whether isolated and/or purified, throughout the specification.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The analogs are designated as 7,17-diHDHAs. In certain aspects, the chiral carbon atom at the 7 position (C-7) has an R configuration. In another aspect, the C-7 carbon atom preferably has an S configuration. In still another aspect, the C-7 carbon atom is as an R/S racemate. Additionally, the chiral carbon atom at the 17 position (C-17) can have an R configuration. Alternatively, the C-17 carbon can have an S configuration. In still yet another aspect, the C-17 carbon can preferably exist as an R/S racemate. Exemplary analogs include, for example, 7S,17R/S-diHDHA, 7S,17R/S-dihydroxy-docosa-4Z,8E,10Z,13Z,15E,19Z-hexaenoic acid.

The present invention further provides compounds and pharmaceutical compositions useful for the treatment of NV, hemangiogenesis and/or angiogenic conditions, having the formula:

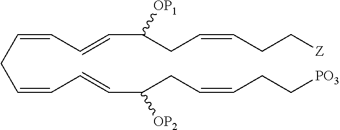

P₁, P₂, P₃ and Z are as defined above.

In certain embodiments, P₁, P₂ and P₃ each are hydrogen atoms and Z is a carboxylic acid or a carboxylic ester. In certain embodiments, when P₁, P₂ and P₃ are hydrogen atoms and Z is a carboxylic acid, the compound is either isolated and/or purified.

In certain aspects the designation of OP₃ serves to denote that the terminal carbon is substituted with one or more halogens (I, Cl, F, Br, mono, di or tri substitution) to form, for example, a trifluoromethyl group, or is an aryl group or phenoxy group that can be substituted or unsubstituted as described herein.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

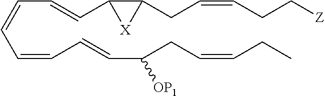

P₁ and Z are as defined above.

X is a substituted or unsubstituted methylene, an oxygen atom, a substituted or unsubstituted nitrogen atom, or a sulfur atom.

In one embodiment, P₁ is a hydrogen atom, X is an oxygen atom and Z is a carboxylic acid or a carboxylic ester. In certain embodiments, when P₁ is a hydrogen atom and Z is a carboxylic acid, the compound is either isolated and/or purified.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

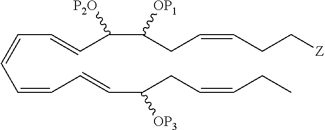

P₁, P₂, P₃ and Z are as defined above.

The analogs are designated as 7,8,17-trihydroxy-DHAs. In certain embodiments, the chiral carbon atom at the 7 position (C-7) has an R configuration. In other embodiments, the C-7 carbon atom preferably has an S configuration. In still other embodiments, the C-7 carbon atom is as an R/S racemate. In certain aspects, the chiral carbon atom at the 8 position (C-8) has an R configuration. In another aspect, the C-8 carbon atom has an S configuration. In still another aspect, the C-8 carbon atom preferably is as an R/S racemate. Additionally, the chiral carbon atom at the 17 position (C-17) can have an R configuration. Alternatively, the C-17 carbon can preferably have an S configuration. In still yet another aspect, the C-17 carbon can exist as an R/S racemate.

In a particular embodiment, P₁, P₂ and P₃ each are hydrogen atoms and Z is a carboxylic acid or a carboxylic ester. In certain embodiments, when P₁, P₂ and P₃ are hydrogen atoms and Z is a carboxylic acid, the compound is either isolated and/or purified.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

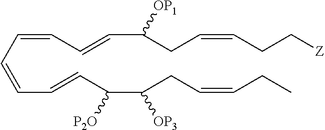

P₁, P₂, P₃ and Z are as defined above.

In a particular embodiment, P₁, P₂ and P₃ each are hydrogen atoms and Z is a carboxylic acid or a carboxylic ester. In certain embodiments, when P₁, P₂ and P₃ are hydrogen atoms and Z is a carboxylic acid, the compound is either isolated and/or purified.

The analogs are designated as 7,16,17-trihydroxy-DHAs. In certain embodiments, the chiral carbon atom at the 7 position (C-7) has an R configuration. In other embodiments, the C-7 carbon atom preferably has an S configuration. In still other embodiments, the C-7 carbon atom is as an R/S racemate. In certain aspects, the chiral carbon atom at the 16 position (C-16) has an R configuration. In another aspect, the C-16 carbon atom has an S configuration. In still another aspect, the C-16 carbon atom preferably is as an R/S racemate. Additionally, the chiral carbon atom at the 17 position (C-17) can have an R configuration. Alternatively, the C-17 carbon can preferably have an S configuration. In still yet another aspect, the C-17 carbon can exist as an R/S racemate.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

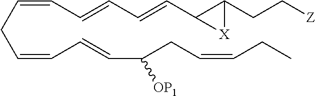

P₁, X and Z are as defined above.

In one embodiment, P₁ is a hydrogen atom, X is an oxygen atom and Z is a carboxylic acid or a carboxylic ester. In certain embodiments, when P₁ is a hydrogen atom and Z is a carboxylic acid, the compound is either isolated and/or purified.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

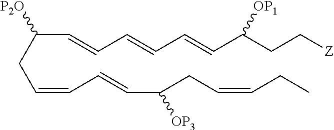

P₁, P₂, P₃ and Z are as defined above.

In a particular embodiment, P₁, P₂ and P₃ each are hydrogen atoms and Z is a carboxylic acid or a carboxylic ester. In certain embodiments, when P₁, P₂ and P₃ are hydrogen atoms and Z is a carboxylic acid, the compound is either isolated and/or purified.

The analogs are designated as 4,11,17-trihydroxy-DHAs. In certain embodiments, the chiral carbon atom at the 4 position (C-4) has an R configuration. In other embodiments, the C-4 carbon atom preferably has an S configuration. In still other embodiments, the C-4 carbon atom is as an R/S racemate. In certain aspects, the chiral carbon atom at the 11 position (C-11) has an R configuration. In another aspect, the C-11 carbon atom has an S configuration. In still another aspect, the C-11 carbon atom preferably is as an R/S racemate. Additionally, the chiral carbon atom at the 17 position (C-17) can have an R configuration. Alternatively, the C-17 carbon can preferably have an S configuration. In still yet another aspect, the C-17 carbon can exist as an R/S racemate.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention provides compounds and pharmaceutical compositions useful for the treatment of NV, hemangiogenesis and/or angiogenic conditions, having the formula:

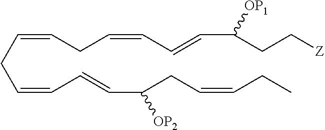

P₁, P₂ and Z are as defined above.

In certain embodiments, P₁ and P₂ are hydrogen atoms and Z is carboxylic acid or a carboxylic ester. In certain embodiments, when P₁, and P₂ are hydrogen atoms and Z is a carboxylic acid, the compound is either isolated and/or purified.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

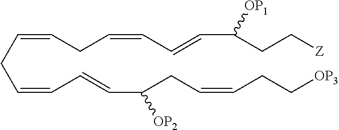

P₁, P₂, P₃ and Z are as defined above.

In a particular embodiment, P₁, P₂ and P₃ each are hydrogen atoms and Z is a carboxylic acid or a carboxylic ester. In certain embodiments, when P₁, P₂ and P₃ are hydrogen atoms and Z is a carboxylic acid, the compound is either isolated and/or purified.

In certain aspects the designation of OP₃ serves to denote that the terminal carbon is substituted with one or more halogens (I, Cl, F, Br, mono, di or tri substitution) to form, for example, a trifluoromethyl group, or is an aryl group or phenoxy group that can be substituted or unsubstituted as described herein.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

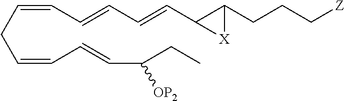

P₁, X and Z are as defined above.

In one embodiment P₁ is a hydrogen atom, X is an oxygen atom and Z is a carboxylic acid or a carboxylic ester. In certain embodiments, when P₁ is a hydrogen atom and Z is a carboxylic acid, the compound is either isolated and/or purified.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention provides compounds and pharmaceutical compositions useful for the treatment of NV, hemangiogenesis and/or angiogenic conditions, having the formula:

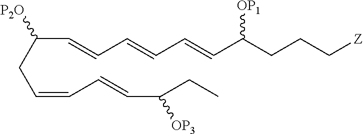

P₁, P₂, P₃ and Z are as defined above.

In one embodiment, P₁, P₂ and P₃ each are hydrogen atoms and Z is a carboxylic acid or a carboxylic ester. In certain embodiments, when P₁, P₂ and P₃ are hydrogen atoms and Z is a carboxylic acid, the compound is either isolated and/or purified.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention provides compounds and pharmaceutical compositions useful for the treatment of NV, hemangiogenesis and/or angiogenic conditions, having the formula:

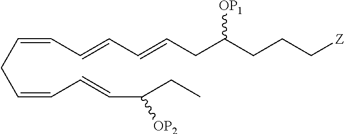

P₁, P₂ and Z are as defined above.

In one particular embodiment, P₁ and P₂ are hydrogen atoms and Z is carboxylic acid or a carboxylic ester. In certain embodiments, when P₁ and P₂ are hydrogen atoms and Z is a carboxylic acid, the compound is either isolated and/or purified.

In one particular embodiment, the compound is 5S, 18 (+/−)-diHEPA, wherein the carboxyl group can be an acid, ester or salt. 5S, 18 (+/−)-diHEPA has been synthesized with 5-lipoxygenase potato and its physical properties are based on LC-MS-MS. Additionally, 5S, 18 (+/−)-diHEPA is biologically active and has anti-inflammatory activity as noted by the downregulation of PMN infiltration in a peritonitis model. It is equipotent to Resolvin E1 at an equal dose amount.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

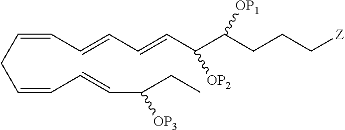

P₁, P₂, P₃ and Z are as defined above.

In one embodiment, P₁, P₂ and P₃ each are hydrogen atoms and Z is a carboxylic acid or a carboxylic ester. In certain embodiments, when P₁, P₂ and P₃ are hydrogen atoms and Z is a carboxylic acid, the compound is either isolated and/or purified.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention also provides compounds and pharmaceutical compositions useful for the treatment of NV, hemangiogenesis and/or angiogenic conditions, having the formula:

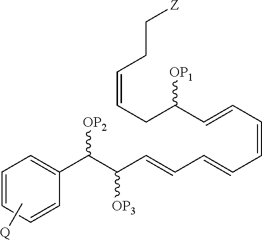

P₁, P₂, P₃, Q and Z are as defined above.

In one particular aspect, P₁, P₂ and P₃ each are hydrogen atoms, each Q is a hydrogen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

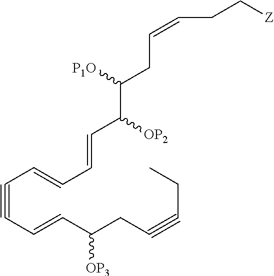

P₁, P₂, P₃ and Z are as defined above.

In one embodiment, P₁, P₂ and P₃ each are hydrogen atoms and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention still further provides compounds and pharmaceutical compositions useful for the treatment of NV, hemangiogenesis and/or angiogenic conditions, having the formula:

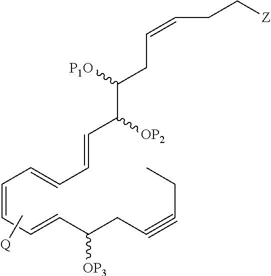

P₁, P₂, P₃, Q and Z are as defined above.

In one particular embodiment, P₁, P₂ and P₃ each are hydrogen atoms, each Q is a hydrogen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

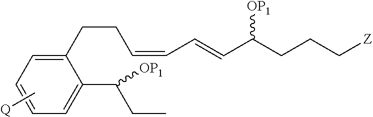

P₁, P₂, Q and Z are as defined above.

In one embodiment, P₁, and P₂ each are hydrogen atoms, each Q is a hydrogen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

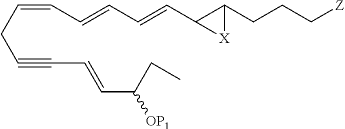

P₁, X and Z are as defined above.

In one aspect, P₁ is a hydrogen atom, X is an oxygen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

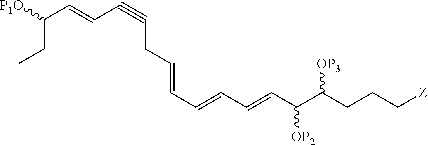

P₁, P₂, P₃ and Z are as defined above.

In one embodiment, P₁, P₂ and P₃ each are hydrogen atoms and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention also provides compounds and pharmaceutical compositions useful for the treatment of NV, hemangiogenesis and/or angiogenic conditions, having the formula:

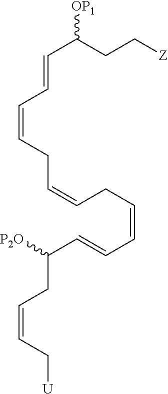

P₁, P₂, U and Z are as defined above.

In one aspect, P₁, and P₂ each are hydrogen atoms, U is a trifluoromethyl group and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of NV, hemangiogenesis and/or angiogenic conditions, having the formula:

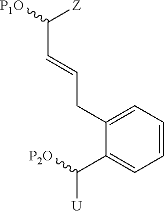

P₁, P₂, U and Z are as defined above. For example, P₁, and P₂ each are hydrogen atoms, U is a trifluoromethyl group and Z is a carboxylic acid or a carboxylic ester.

The present invention, further pertains to dihydroxy-docosahexaenoic acid analogs useful to treat NV, hemangiogenesis and/or angiogenic conditions (diHDHA) having the formula

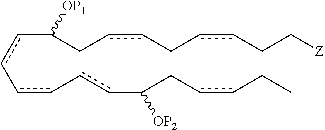

The analogs are designated as 10,17-diHDHAs. P₁ and P₂ are as defined above and can be the same or different. Z is as defined above and in particular can be a carboxylic acid, ester, amide, thiocarbamate, carbamate, thioester, thiocarboxamide or a nitrile. The broken double bond line indicates that either the E or Z isomer is within the scope of the analog(s). In certain aspects, the chiral carbon atom at the 10 position (C-10) has an R configuration. In another aspect, the C-10 carbon atom has an S configuration. In still another aspect, the C-10 carbon atom preferably is as an R/S racemate. Additionally, the chiral carbon atom at the 17 position (C-17) can have an R configuration. Alternatively, the C-17 carbon can preferably have an S configuration. In still yet another aspect, the C-17 carbon can exist as an R/S racemate. In one example, the present invention includes 10,17S-docosatriene, 10,17-dihydroxy-docosa-4Z,7Z,11E,13,15E,19Z-hexaenoic acid analogs such as 10R/S-OCH_{3,17}S-HDHA, 10R/S, methoxy-17 S hydroxy-docosa-4Z,7Z,11E,13,15E,19Z-hexaenoic acid derivatives.

In certain embodiments, when P₁ and P₂ are hydrogen atoms and Z is a carboxylic acid, the compound is either isolated and/or purified.

In still yet another embodiment, the present invention pertains to diHDHA analogs useful to treat NV, hemangiogenesis and/or angiogenic conditions having the formula

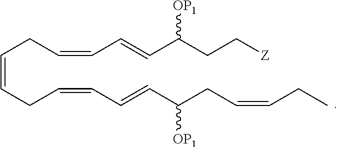

The analogs are designated as 4,17-diHDHAs. P₁, P₂ and Z are as defined above. P₁ and P₂ can be the same or different. In particular, Z can be a carboxylic acid, ester, amide, thiocarbamate, carbamate, thioester, thiocarboxamide or a nitrile. In certain aspects, the chiral carbon atom at the 4 position (C-4) has an R configuration. In another aspect, the C-4 carbon atom preferably has an S configuration. In still another aspect, the C-4 carbon atom is as an R/S racemate. Additionally, the chiral carbon atom at the 17 position (C-17) can have an R configuration. Alternatively, the C-17 carbon can have an S configuration. In still yet another aspect, the C-17 carbon can preferably exist as an R/S racemate.

In certain embodiments, when P₁ and P₂ are hydrogen atoms and Z is a carboxylic acid, the compound is either isolated and/or purified.

For example, the present invention includes 4S, 17R/S-diHDHA, 4S,17R/S-dihydroxy-docosa-5E,7Z,10Z,13Z,15E,19Z-hexaenoic acid analogs.

It should be understood that “Z” can be altered from one particular moiety to another by a skilled artisan. In order to accomplish this in some particular instances, one or more groups may require protection. This is also within the skill of an ordinary artisan. For example, a carboxylic ester (Z) can be converted to an amide by treatment with an amine. Such interconversion are known in the art.

In the EPA and DHA analogs, it should be understood that reference to “hydroxyl” stereochemistry is exemplary, and that the term is meant to include protected hydroxyl groups as well as the free hydroxyl group. In certain embodiments, the C-17 position has an R configuration. In other embodiment, the C-17 position has an S configuration. In other aspects, certain embodiments of the invention have an R configuration at the C-18 position.

In certain aspects of the present invention, ASA pathways generate R>S and therefore, 4S, 5R/S, 7S,8R/S, 11R, 12R/S 16 S, 17 R. With respect to species generated from the 15-LO pathway the chirality of C-17 is S, C-16 R and C-10, preferably R.

The hydroxyl(s) in the EPA and DHA analogs can be protected by various protecting groups (P), such as those known in the art. An artisan skilled in the art can readily determine which protecting group(s) may be useful for the protection of the hydroxyl group(s). Standard methods are known in the art and are more fully described in literature. For example, suitable protecting groups can be selected by the skilled artisan and are described in Green and Wuts, “Protecting Groups in Organic Synthesis”, John Wiley and Sons, Chapters 5 and 7, 1991, the teachings of which are incorporated herein by reference. Preferred protecting groups include methyl and ethyl ethers, TMS or TIPPS groups, acetate (esters) or propionate groups and glycol ethers, such as ethylene glycol and propylene glycol derivatives.

For example, one or more hydroxyl groups can be treated with a mild base, such as triethylamine in the presence of an acid chloride or silyl chloride to facilitate a reaction between the hydroxyl ion and the halide. Alternatively, an alkyl halide can be reacted with the hydroxyl ion (generated by a base such as lithium diisopropyl amide) to facilitate ether formation.

The compounds can be prepared by methods provided in U.S. patent application Ser. No. 09/785,866, filed Feb. 16, 2001, entitled “Aspirin Triggered Lipid Mediators” by Charles N. Serhan and Clary B. Clish, U.S. patent application Ser. No. 10/639,714, filed Aug. 12, 2003, entitled “Resolvins: Biotemplates for Novel Therapeutic Interventions” by Charles N. Serhan, PCT Applications WO 01/60778, filed Feb. 16, 2001, entitled “Aspirin Triggered Lipid mediators” by Charles N. Serhan and Clary B. Clish and WO 04/014835, filed Aug. 12, 2003, entitled “Resolvins: Biotemplates for Novel Therapeutic Interventions” by Charles N. Serhan, and US Publication No. 2004/0044050, entitled “Analogues of Lipic Mediators Derived from Omega-3 PUFAs and Methods of Use”, by Daniel Goodman et al., the contents of which are incorporated herein by reference in their entirety for all purposes.

It should also be understood that for the EPA and DHA analogs, not all hydroxyl groups need be protected. One, two or all three hydroxyl groups can be protected. This can be accomplished by the stoichiometric choice of reagents used to protect the hydroxyl groups. Methods known in the art can be used to separate the di- or tri-protected hydroxy compounds, e.g., HPLC, LC, flash chromatography, gel permeation chromatography, crystallization, distillation, etc.

It should be understood that there are one or more chiral centers in each of the above-identified compounds. It should understood that the present invention encompasses all stereochemical forms, e.g., enantiomers, diastereomers and racemates of each compound. Where asymmetric carbon atoms are present, more than one stereoisomer is possible, and all possible isomeric forms are intended to be included within the structural representations shown. Optically active (R) and (S) isomers may be resolved using conventional techniques known to the ordinarily skilled artisan. The present invention is intended to include the possible diastereiomers as well as the racemic and optically resolved isomers.

The resolvin analogs depicted throughout the specification contain acetylenic and/or ethylenically unsaturated sites. Where carbon carbon double bonds exist, the configurational chemistry can be either cis (Z) or trans (E) and the depictions throughout the specification are not meant to be limiting. The depictions are, in general, presented based upon the configurational chemistry of related DHA or EPA compounds, and although not to be limited by theory, are believed to possess similar configuration chemistry.

Throughout the specification carbon carbon bonds in particular have been “distorted” for ease to show how the bonds may ultimately be positioned relative one to another. For example, it should be understood that acetylenic portions of the resolvins actually do include a geometry of approximately 180 degrees, however, for aid in understanding of the synthesis and relationship between the final product(s) and starting materials, such angles have been obfuscated to aid in comprehension.

It should be understood that hydrogenation of acetylenic portions of the resolvin analog may result in one or more products.

It is intended that all possible products are included within this specification. For example, hydrogenation of a diacetylenic resolvin analog can produce up to 8 products (four diene products, i.e., cis, cis; cis, trans; trans, cis; trans, trans) if hydrogenation of both acetylenic portions is completed (this can be monitored by known methods) and four monoacetylene-monoethylene products (cis or trans “monoene”-acetylene; acetylene-cis or trans “monoene”. All products can be separated and identified by HPLC, GC, MS, NMR, IR.

Known techniques in the art can be used to convert the carboxylic acid/ester functionality of the resolvin analog into carboxamides, thioesters, nitrile, carbamates, thiocarbamates, etc. and are incorporated herein. The appropriate moieties, such as amides, can be further substituted as is known in the art.

In one aspect of the invention, the compound(s) of the invention are substantially purified and isolated by techniques known in the art. The purity of the purified compounds is generally at least about 90%, preferably at least about 95%, and most preferably at least about 99% by weight.

In general, the resolvin analogs of the invention are bioactive as alcohols. Enzymatic action or reactive oxygen species attack at the site of inflammation or degradative metabolism. Such interactions with the hydroxyl(s) of the resolvin molecule can eventually reduce physiological activity as depicted below:

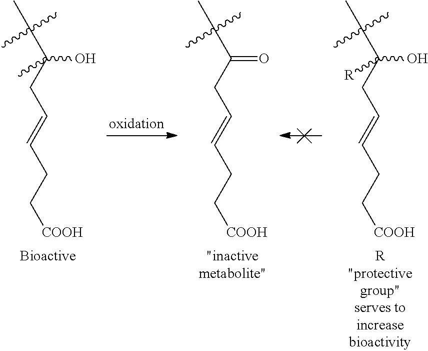

The use of “R” groups with secondary bioactive alcohols, in particular, serves to increase the bioavailability and bioactivity of the resolvin analog by inhibiting or diminishing the potential for oxidation of the alcohol to a ketone producing an inactive metabolite. The R “protecting groups” include, for example, linear and branched, substituted and unsubstituted alkyl groups, aryl groups, alkylaryl groups, phenoxy groups, and halogens.

Generally the use of “R protection chemistry” is not necessary with vicinal diols within the resolvin analog. Typically vicinal diols are not as easily oxidized and therefore, generally do not require such protection by substitution of the hydrogen atom adjacent to the oxygen atom of the hydroxyl group. Although it is generally considered that such protection is not necessary, it is possible to prepare such compounds where each of the vicinal diol hydroxyl groups, independently, could be “protected” by the substitution of the hydrogen atom adjacent to the oxygen atom of the hydroxyl group with an “R protecting group” as described above.

The term “tissue” is intended to include intact cells, blood, blood preparations such as plasma and serum, bones, joints, muscles, smooth muscles, and organs.

The term “subject” is intended to include living organisms susceptible to conditions or diseases caused or contributed bacteria, pathogens, disease states or conditions as generally disclosed, but not limited to, throughout this specification. Examples of subjects include humans, dogs, cats, cows, goats, and mice. The term subject is further intended to include transgenic species.

When the compounds of the present invention are administered as pharmaceuticals, to humans and mammals, they can be given per se or as a pharmaceutical composition containing, for example, 0.1 to 99.5% (more preferably, 0.5 to 90%) of active ingredient, i.e., at least one EPA or DHA analog, in combination with a pharmaceutically acceptable carrier.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a compound(s) of the present invention within or to the subject such that it can perform its intended function. Typically, such compounds are carried or transported from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

In certain embodiments, the compounds of the present invention may contain one or more acidic functional groups and, thus, are capable of forming pharmaceutically acceptable salts with pharmaceutically acceptable bases. The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M., et al., “Pharmaceutical Salts,” J. Pharm. Sci., 1977; 66:1-19 which is incorporated herein by reference).

The term “pharmaceutically acceptable esters” refers to the relatively non-toxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. The term is further intended to include lower hydrocarbon groups capable of being solvated under physiological conditions, e.g., alkyl esters, methyl, ethyl and propyl esters.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for intravenous, oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention. Such solutions are useful for the treatment of the conditions described herein, such as NV, hemangiogenesis or angiogenesis of the corneal tissue.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Intravenous injection administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systematically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of ordinary skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 0.1 to about 40 mg per kg per day. For example, between about 0.01 microgram and 20 micrograms, between about 20 micrograms and 100 micrograms and between about 10 micrograms and 200 micrograms of the compounds of the invention are administered per 20 grams of subject weight.

If desired, the effective daily dose of the active compound may be administered as two, three, four, five, six or more sub-doses administered separately at appropriate intervals throughout the day, optionally, in unit dosage forms.

The pharmaceutical compositions of the invention include a “therapeutically effective amount” or a “prophylactically effective amount” of one or more of the EPA or DHA analogs of the invention. A “therapeutically effective amount” refers to an amount effective, at dosages and for periods of time necessary, to achieve the desired therapeutic result, e.g., a diminishment or prevention of effects associated with various disease states or conditions. A therapeutically effective amount of the EPA or DHA analog may vary according to factors such as the disease state, age, sex, and weight of the individual, and the ability of the therapeutic compound to elicit a desired response in the individual. A therapeutically effective amount is also one in which any toxic or detrimental effects of the therapeutic agent are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the EPA or DHA analog and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a EPA or DHA analog of the invention is 0.1-20 mg/kg, more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

While it is possible for a compound of the present invention to be administered alone, it is preferable to administer the compound as a pharmaceutical composition.

The invention features an article of manufacture that contains packaging material and a EPA or DHA analog formulation contained within the packaging material. This formulation contains an at least one EPA or DHA analog and the packaging material contains a label or package insert indicating that the formulation can be administered to the subject to treat one or more conditions as described herein, in an amount, at a frequency, and for a duration effective to treat or prevent such condition(s). Such conditions are mentioned throughout the specification and are incorporated herein by reference. Suitable EPA analogs and DHA analogs are described herein.

More specifically, the invention features an article of manufacture that contains packaging material and at least one EPA or DHA analog contained within the packaging material. The packaging material contains a label or package insert indicating that the formulation can be administered to the subject to asthma in an amount, at a frequency, and for a duration effective treat or prevent symptoms associated with such disease states or conditions discussed throughout this specification.

### REFERENCES

1. Edelman J L, Castro M R, Wen Y. Correlation of VEGF expression by leukocytes with the growth and regression of blood vessels in the rat cornea. Invest Ophthalmol Vis Sci. 1999; 40:1112-1123.
2. Naldini A, Carraro F. Role of inflammatory mediators in angiogenesis. Curr Drug Targets Inflamm Allergy. 2005; 4:3-8.
3. Benelli R, Morini M, Carrozzino F, et al. Neutrophils as a key cellular target for angiostatin: implications for regulation of angiogenesis and inflammation. FASEB J. 2002; 16:267-269.
4. Sterescu A E, Rousseau-Harsany E, Farrell C, Powell J, David M, Dubois J. The potential efficacy of omega-3 fatty acids as anti-angiogenic agents in benign vascular tumors of infancy. Med Hypotheses. 2006; 66:1121-1124.
5. Serhan C N, Chiang N, Van Dyke T E. Resolving inflammation: dual anti-inflammatory and pro-resolution lipid mediators. Nat Rev Immunol. 2008; 8:349-361.
6. Serhan C N, Clish C B, Brannon J, Colgan S P, Chiang N, Gronert K. Novel functional sets of lipid-derived mediators with antiinflammatory actions generated from omega-3 fatty acids via cyclooxygenase 2-nonsteroidal antiinflammatory drugs and transcellular processing. J Exp Med. 2003; 192:1197-1204.
7. Serhan C N, Hong S, Gronert K, et al. Resolvins: a family of bioactive products of omega-3 fatty acid transformation circuits initiated by aspirin treatment that counter proinflammation signals. J Exp Med. 2002; 196:1025-1037.
8. Brink C, Dahlén S E, Drazen J, et al. International Union of Pharmacology XXXVII. Nomenclature for leukotriene and lipoxin receptors. Pharmacol Rev. 2003; 55:195-222.
9. Munger K A, Montero A, Fukunaga M, et al. Transfection of rat kidney with human 15-lipoxygenase suppresses inflammation and preserves function in experimental glomerulonephritis. Proc Natl Acad Sci. 1999; 96:13375-13380.
10. Bandeira-Melo C, Bozza P T, Diaz B L, et al. Cutting edge: Lipoxin (LX) A4 and aspirin-triggered 15-epi-LXA4 block allergen-induced eosinophil trafficking. J Immunol. 2002; 164:2267-2271.
11. Levy B D, De Sanctis G T, Devchand P R, et al. Multi-pronged inhibition of airway hyper-responsiveness and inflammation by lipoxin A4. Nat Med. 2002; 8:1018-1023.
12. Karp C L, Flick L M, Park K W, et al. Defective lipoxin-mediated anti-inflammatory activity in the cystic fibrosis airway. Nat Immunol. 2004; 5:388-392.
13. Serhan C N, Jain A, Marleau S, et al. Reduced inflammation and tissue damage in transgenic rabbits overexpressing 15-lipoxygenase and endogenous anti-inflammatory lipid mediators. J Immunol. 2003; 71:6856-6865.
14. Dana M R, Zhu S N, Yamada J. Topical modulation of interleukin-1 activity in corneal neovascularization. Cornea. 1998; 17:403-409.
15. Dana M R, Streilein J W. Loss and restoration of immune privilege in eyes with corneal neovascularization. Invest Ophthalmol Vis Sci. 1996; 37:2485-2494.
16. Williams C S, Tsujii M, Reese J, Dey S K, DuBois R N. Host cyclooxygenase-2 modulates carcinoma growth. J Clin Invest. 2005; 105:1589-1594.
17. Põld M, Zhu L X, Sharma S, et al. Cyclooxygenase-2-dependent expression of angiogenic CXC chemokines ENA-78/CXC ligand (CXCL) 5 and interleukin-8/CXCL8 in human non-small cell lung cancer. Cancer Res. 2004; 64:1853-1860.
18. Arita M, Bianchini F, Aliberti J, et al. Stereochemical assignment, antiinflammatory properties, and receptor for the omega-3 lipid mediator resolvin E1. J Exp Med. 2005; 201:713-722.
19. Sun Y P, Oh S F, Uddin J, et al. Resolvin D1 and its aspirin-triggered 17R epimer. Stereochemical assignments, anti-inflammatory properties, and enzymatic inactivation. J Biol Chem. 2007; 282:9323-9334.
20. Serhan C N, Maddox J F, Petasis N A, et al. Design of lipoxin A4 stable analogs that block transmigration and adhesion of human neutrophils. Biochemistry. 1995; 34:14609-14615.
21. Jin Y, Shen L, Chong E M, et al. The chemokine receptor CCR7 mediates corneal antigen-presenting cell trafficking. Mol Vis. 2007; 13:626-634.
22. Deshpande S, Zheng M, Lee S, et al. Bystander activation involving T lymphocytes in herpetic stromal keratitis. J Immunol. 2001; 167:2902-2910.
23. Biswas P S, Banerjee K, Zheng M, et al. Counteracting corneal immunoinflammatory lesion with interleukin-1 receptor antagonist protein. J Leukoc Biol. 2004; 76:868-875.
24. Sonoda K, Sakamoto T, Yoshikawa H, et al. Inhibition of corneal inflammation by the topical use of Ras farnesyltransferase inhibitors: selective inhibition of macrophage localization. Invest Ophthalmol Vis Sci. 1998; 39:2245-2251.
25. Serhan C N, Savill J. Resolution of inflammation: the beginning programs the end. Nat Immunol. 2005; 12:1191-1197.
26. Schwab J M, Chiang N, Arita M, Serhan C N. Resolvin E1 and protectin D1 activate inflammation-resolution programmes. Nature. 2007; 447:869-874.
27. Torisu H, Ono M, Kiryu H, et al. Macrophage infiltration correlates with tumor stage and angiogenesis in human malignant melanoma: possible involvement of TNFalpha and IL-1 alpha. Int J Cancer. 2000; 85:182-188.
28. Ryuto M, Ono M, Izumi H, et al. Induction of vascular endothelial growth factor by tumor necrosis factor alpha in human glioma cells. Possible roles of SP-1. J Biol Chem. 1996; 271:28220-28228.
29. Yoshida S, Ono M, Shono T, et al. Involvement of interleukin-8, vascular endothelial growth factor, and basic fibroblast growth factor in tumor necrosis factor alpha-dependent angiogenesis. Mol Cell Biol. 1997; 17:4015-4023.
30. Joukov V, Pajusola K, Kaipainen A, et al. A novel vascular endothelial growth factor, VEGF-C, is a ligand for the Flt4 (VEGFR-3) and KDR (VEGFR-2) receptor tyrosine kinases. EMBO J. 1996; 15:290-298.
31. Baldwin M E, Roufail S, Halford M M, Alitalo K, Stacker S A, Achen M G. Multiple forms of mouse vascular endothelial growth factor-D are generated by RNA splicing and proteolysis. J Biol Chem. 2001; 276:44307-44414.
32. Fierro I M. Angiogenesis and lipoxins. Prostaglandins Leukot Essent Fatty Acids. 2005; 73:271-275.
33. Connor K M, SanGiovanni J P, Lofqvist C, et al. Increased dietary intake of omega-3-polyunsaturated fatty acids reduces pathological retinal angiogenesis. Nat Med. 2007; 13:868-873.
34. Nakao S, Kuwano T, Tsutsumi-Miyahara C, et al. Infiltration of COX-2-expressing macrophages is a prerequisite for IL-1 beta-induced neovascularization and tumor growth. J Clin Invest. 2005; 115:2979-2991.
35. Fierro I M, Kutok J L, Serhan C N. Novel lipid mediator regulators of endothelial cell proliferation and migration: aspirin-triggered-15R-lipoxin A(4) and lipoxin A(4). J Pharmacol Exp Ther. 2002; 300:385-392.
36. Clish C B, O'Brien J A, Gronert K, Stahl G L, Petasis N A, Serhan C N. Local and systemic delivery of a stable aspirin-triggered lipoxin prevents neutrophil recruitment in vivo. Proc Natl Acad Sci. 1999; 96:8247-8252.
37. Chiang N, Serhan C N, Dahlen S E, et al. The lipoxin receptor ALX: potent ligand-specific and stereoselective actions in vivo. Pharmacol Rev. 2006; 58:463-487.
38. Cezar-de-Mello P F, Nascimento-Silva V, Villela C G, Fierro I M. Aspirin-triggered Lipoxin A4 inhibition of VEGF-induced endothelial cell migration involves actin polymerization and focal adhesion assembly. Oncogene. 2006; 25:122-129.
39. Cezar-de-Mello P F, Vieira A M, Nascimento-Silva V, Villela C G, Barja-Fidalgo C, Fierro I M. ATL-1, an analogue of aspirin-triggered lipoxin A(4), is a potent inhibitor of several steps in angiogenesis induced by vascular endothelial growth factor. Br J Pharmacol. 2008; 153:956-965.
40. Wittamer V, Franssen J D, Vulcano M, et al. Specific recruitment of antigen-presenting cells by chemerin, a novel processed ligand from human inflammatory fluids. J Exp Med. 2003; 198:977-985.
41. Gronert K, Gewirtz A, Madara J L, Serhan C N. Identification of a human enterocyte lipoxin A4 receptor that is regulated by interleukin (IL)-13 and interferon gamma and inhibits tumor necrosis factor alpha-induced IL-8 release. J Exp Med. 1998; 187:1285-1294.
42. Gronert K, Maheshwari N, Khan N, Hassan I R, Dunn M, Laniado Schwartzman M. A role for the mouse 12/15-lipoxygenase pathway in promoting epithelial wound healing and host defense. J Biol Chem. 2005; 280:15267-15278.

Although the present invention has been described with reference to preferred embodiments, persons skilled in the art will recognize that changes may be made in form and detail without departing from the spirit and scope of the invention. All publications and references cited herein, including those in the background section, are expressly incorporated herein by reference in their entirety.

What is claimed is:

1. A method to treat or prevent neovascularization, comprising the step of treating corneal tissue with compound comprising the formula: 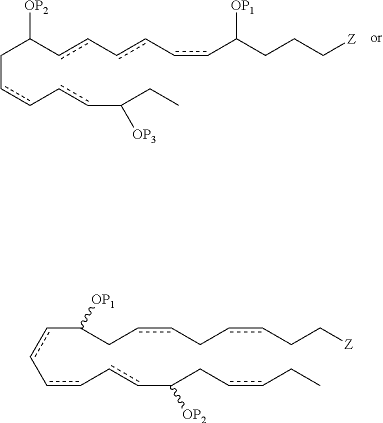
wherein a bond depicted as [symbol] represents either a cis or trans double bond;
each P₁, P₂ and P₃, individually are protecting groups, hydrogen atoms or combinations thereof;
Z is —C(O)OR^{d}, —C(O)NR^{c}R^{c}, —C(O)H, —C(NH)NR^{c}R^{c}, —C(S)H, —C(S)OR^{d}, —C(S)NR^{c}R^{c}, —CN;
each R^{a}, if present, is independently selected from the group consisting of hydrogen, (C1-C6) alkyl, (C3-C8) cycloalkyl, cyclohexyl, (C4-C11) cycloalkylalkyl, (C5-C10) aryl, phenyl, (C6-C16) arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;
each R^{b}, if present, is a suitable group independently selected from the group consisting of ═O, —OR^{d}, (C1-C3) haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
each R^{c}, if present, is independently a protecting group or R^{a}, or, alternatively, each R^{c} is taken together with the nitrogen atom to which it is bonded to form a 5 to 8-membered cycloheteroalkyl or heteroaryl which may optionally include one or more of the same or different additional heteroatoms and which may optionally be substituted with one or more of the same or different R^{a} or suitable R^{b} groups;
each n, independently, if present, is an integer from 0 to 3;
each R^{d}, independently, if present, is a protecting group or R^{a}; or
pharmaceutically acceptable salts thereof.

2. A method to treat or prevent hemangiogenesis, comprising the step of treating corneal tissue with compound comprising the formula: 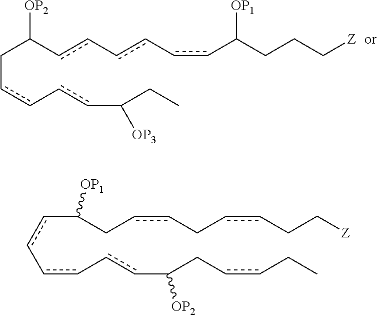
wherein a bond depicted as [symbol] represents either a cis or trans double bond;
each P₁, P₂ and P₃, individually are protecting groups, hydrogen atoms or combinations thereof;
Z is —C(O)OR^{d}, —C(O)NR^{c}R^{c}, —C(O)H, —C(NH)NR^{c}R^{c}, —C(S)H, —C(S)OR^{d}, —C(S)NR^{c}R^{c}, —CN;
each R^{a}, if present, is independently selected from the group consisting of hydrogen, (C1-C6) alkyl, (C3-C8) cycloalkyl, cyclohexyl, (C4-C11) cycloalkylalkyl, (C5-C10) aryl, phenyl, (C6-C16) arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;
each R^{b}, if present, is a suitable group independently selected from the group consisting of ═O, —OR^{d}, (C1-C3) haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
each R^{c}, if present, is independently a protecting group or R^{a}, or, alternatively, each R^{c} is taken together with the nitrogen atom to which it is bonded to form a 5 to 8-membered cycloheteroalkyl or heteroaryl which may optionally include one or more of the same or different additional heteroatoms and which may optionally be substituted with one or more of the same or different R^{a} or suitable R^{b} groups;
each n, independently, if present, is an integer from 0 to 3;
each R^{d}, independently, if present, is a protecting group or R^{a}; or
pharmaceutically acceptable salts thereof.

3. A method to treat or prevent an angiogenic condition of corneal tissue, comprising the step of treating corneal tissue with compound comprising the formula: 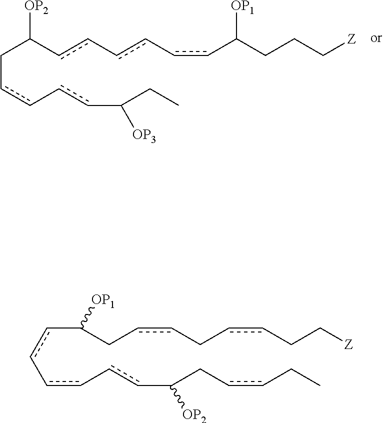
wherein a bond depicted as [symbol] represents either a cis or trans double bond;
each P₁, P₂ and P₃, individually are protecting groups, hydrogen atoms or combinations thereof;
Z is —C(O)OR^{d}, —C(O)NR^{c}R^{c}, —C(O)H, —C(NH)NR^{c}R^{c}, —C(S)H, —C(S)OR^{d}, —C(S)NR^{c}R^{c}, —CN;
each R^{a}, if present, is independently selected from the group consisting of hydrogen, (C1-C6) alkyl, (C3-C8) cycloalkyl, cyclohexyl, (C4-C11) cycloalkylalkyl, (C5-C10) aryl, phenyl, (C6-C16) arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;
each R^{b}, if present, is a suitable group independently selected from the group consisting of ═O, —OR^{d}, (C1-C3) haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
each R^{c}, if present, is independently a protecting group or R^{a}, or, alternatively, each R^{c} is taken together with the nitrogen atom to which it is bonded to form a 5 to 8-membered cycloheteroalkyl or heteroaryl which may optionally include one or more of the same or different additional heteroatoms and which may optionally be substituted with one or more of the same or different R^{a} or suitable R^{b} groups;
each n, independently, if present, is an integer from 0 to 3;
each R^{d}, independently, if present, is a protecting group or R^{a}; or
pharmaceutically acceptable salts thereof.

4. The method of claim 1, further comprising a pharmaceutically acceptable carrier.

5. The method of claim 2, further comprising a pharmaceutically acceptable carrier.

6. The method of claim 3, further comprising a pharmaceutically acceptable carrier.
